(19) United States
(12) Patent Application Publication
(10) Pub. No.: US 2026/0232949 A1
(43) Pub. Date: Aug. 13, 2026

(54) METHODS OF MANAGEMENT OF ATTENTION DISORDERS

(71) Applicant: Vizo Specs Ltd., Tel Aviv (IL)

(72) Inventors: Yoav KAHANE, Tel Aviv (IL); Yael RICHTER, Kibbutz Sarid (IL); Dario GEISINGER, Petach Tikva (IL)

(21) Appl. No.: 19/470,900

(22) PCT Filed: Mar. 31, 2024
(86) PCT No.: PCT/IL2024/050331

§ 371 Date: Sep. 30, 2025

Related U.S. Application Data
(60) Provisional application No. 63/494,044, filed on Apr. 4, 2023.

(51) Int. Cl. A61M 21/00

(52) U.S. Cl. CPC A61M 21/00; A61M 2021/0044; A61M 2209/088

(57) ABSTRACT

The present disclosure provides methods for any one of treating a subject having attention disorder, for selecting a location of a correction coordinate in a device including at least one lens, the correction coordinate lies in the field of view of a subject, when the subject wears the device; or for converting an eyeglass into a device for improving performance of subject having an attention disorder. The methods involve placing a correcting element on a location on the at least one lens, the location of the correction element being within a defined correction region having a shape of two dimensional double arc.

### TECHNOLOGICAL FIELD

The present disclosure relates to management of attention disorder.

### BACKGROUND ART

References considered to be relevant as background to the presently disclosed subject matter are listed below:
U.S. Pat. No. 10,149,798

Acknowledgement of the above references herein is not to be inferred as meaning that these are in any way relevant to the patentability of the presently disclosed subject matter.

### BACKGROUND

Devices and techniques have been developed to correct physiological as well as psychological and cognitive states of patients.

U.S. Pat. No. 10,149,798 describes methods, systems and devices for improving a defined condition in a subject, by selecting a set of one or more correction zones, being angular zones in the subject's field of view, the one or more correction zones being associated with the condition; and placing the one or more correcting elements in said one or more correction zones to thereby cause improvement in said condition.

### GENERAL DESCRIPTION

The present disclosure provides, in accordance with a first of its aspects, a method for assisting a subject having attention disorder in improving the subject's attention performance, and/or for treating a subject having attention disorder, the method comprising administering, to the subject having the attention disorder, a device comprising at least one lens, and at least one correcting element disposed on the at least one lens,
wherein the at least one correcting element is disposed so as to be within the subject's field of view when the device is worn;
wherein the at least one correcting element is positioned at a correction coordinate that is within a correction region;
wherein the correction region and the correction coordinate are defined using a polar coordinate system measured from a lens center of the at least one lens, wherein the lens center is configured to be horizontally aligned with an optical center of a left eye or a right eye of the subject, when the device is worn by the subject;
wherein the correction region has a two-dimensional double arc shape including a proximal arc and a distal arc, each of said proximal arc and distal arc face said lens center,
   wherein the proximal arc extends between a first proximal end and a second proximal end, and said distal arc extends between a first distal end and a second distal end,
   wherein the first proximal end and said first distal are connected by a first transverse line, and said second proximal end and second distal end is connected by a second transverse line,
   wherein at least one point on said proximal arc has a proximal distance from said lens center that is between 4 mm and about 6 mm and at least one point on said distal arc has a distal distance from said lens center that is between about 13 mm and 14 mm; and
   wherein a radial line connecting said first distal end to said lens center defines a first angle that is between 25° and 55° and a radial line connecting said second distal end to said lens center defines a second angle that is between 125° and 155°.

In accordance with a second aspect of the presently disclosed subject matter there is provided a method for selecting a location of a correction coordinate in a device including at least one lens, the correction coordinate lies in the field of view of a subject, when the subject wears the device; the method comprising:
identifying on said at least one lens a correction region,
   wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
   wherein said correction region has a two-dimensional double arc shape including a proximal arc and a distal arc, each of said proximal arc and distal arc face said lens center,
   wherein the proximal arc extends between a first proximal end and a second proximal end, and said distal arc extends between a first distal end and a second distal end,
   wherein the first proximal end and said first distal are connected by a first transverse line, and said second proximal end and second distal end is connected by a second transverse line,
   wherein said proximal arc has a proximal distance from said lens center that is between 4 mm and about 6 mm and said distal arc has a distal distance from said lens center that is between about 13 mm and 14 mm; and
   wherein a radial line connecting said first distal end to said lens center defines a first angle that is between 25° and 55° and a radial line connecting said second distal end to said lens center defines a second angle that is between 125° and 155°; and
selecting said location of said correction coordinate in said correction region for placing thereon a correcting element for improving attention performance in a subject having attention disorder.

In accordance with a third aspect, the presently disclosed subject matter provides a method of converting an eyeglass into a device for improving performance of subject having an attention disorder, and/or for treating a subject having attention disorder, the method comprising:
selecting a location of at least one correction coordinate on at least one lens of said eyeglass, for placing thereon a correcting element, said selecting comprises at least the steps of
   identifying on said at least one lens a correction region,
      wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
      wherein said correction region has a two-dimensional double arc shape including a proximal arc and a distal arc, each of said proximal arc and distal arc face said lens center,
      wherein the proximal arc extends between a first proximal end and a second proximal end, and said distal arc extends between a first distal end and a second distal end;
      wherein the first proximal end and said first distal are connected by a first transverse line, and said second proximal end and second distal end is connected by a second transverse line;
      wherein said proximal arc has a proximal distance from said lens center that is between 4 mm and about 6 mm and said distal arc has a distal distance from said lens center that is between about 13 mm and 14 mm; and
      wherein a radial line connecting said first distal end to said lens center defines a first angle that is between 25° and 55° and a radial line connecting said second distal end to said lens center defines a second angle that is between 125° and 155°; and
   selecting said location of said correction coordinate in said correction region, the correction coordinate being within the subject's field of view when the eyeglass is worn by the subject; and
placing said at least one correcting element in the selected location in said correction coordinate.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a front view schematic illustration of eyeglasses marked with a polar coordinate system for placing correcting elements in accordance with the present disclosure.

FIGS. 2A-2F are schematic illustrations of a front view of eyeglasses marked with a polar coordinate system and illustrating different alternatives for a double arc shape of a correction region of the presently disclosed subject matter.

FIGS. 3A-3B are schematic illustrations of right lens (FIG. 3A) and left lens (FIG. 3B) holding correcting elements according to the non-limiting Examples 1 to 15, the location of correcting elements being defined by a polar coordinate system, according to the presently disclosed subject matter.

FIGS. 4A-4B are schematic illustrations of right lens (FIG. 4A) and left lens (FIG. 4B) and location of correcting elements according to additional examples of the presently disclosed subject matter, which are not included in FIGS. 3A-3B.

### DETAILED DESCRIPTION

The presently disclosed subject matter is based on the development of the identification of a unique and well-defined region within eyeglass lens(es) that is associated with improving performance of individuals with a pre-existing Attention Deficit Hyperactivity Disorder (ADHD).

Specifically, it has been found that placing an interference in a subject's field of view, the interference being any type of a mark (such as a sticker, an opaque mark, a color mark, an etch, electronic, etc.) within the presently disclosed attention disorder-associated correction region, results in improvement in the subject's attention according to at least one test acceptable by the practitioners, as further detailed below.

Thus, in accordance with a first aspect of the presently disclosed subject matter there is provided a method for assisting a subject having attention disorder in improving the subject's attention performance, and/or for treating a subject having an attention disorder, the method comprising administering, to the subject having pre-existing attention disorder, a device comprising at least one lens, and at least one correcting element disposed on the at least one lens,
wherein the at least one correcting element is disposed so as to be within the subject's field of view when the device is worn;
wherein the at least one correcting element is positioned at a correction coordinate that is within a correction region;
wherein the correction coordinate and the correction regions are each defined using a polar coordinate system measured from a lens center of the at least one lens, wherein the lens center is configured to be horizontally aligned with an optical center of a left eye or a right eye of the subject, when the device is worn by the subject;
wherein the correction region has a two-dimensional double arc shape including a proximal arc and a distal arc, each of said proximal arc and distal arc face said lens center,
wherein the proximal arc extends between a first proximal end and a second proximal end, and said distal arc extends between a first distal end and a second distal end,
wherein the first proximal end and said first distal are connected by a first transverse line, and said second proximal end and second distal end is connected by a second transverse line,
wherein at least one point on said proximal arc has a proximal distance from said lens center that is between 4 mm and about 6 mm and at least one point on said distal arc has a distal distance from said lens center that is between about 13 mm and 14 mm; and
wherein a radial line connecting said first distal end to said lens center defines a first angle that is between 25° and 55° and a radial line connecting said second distal end to said lens center defines a second angle that is between 125° and 155°.

In accordance with a second aspect of the presently disclosed subject matter there is provided a method for selecting a location of a correction coordinate in a device including at least one lens, the correction coordinate lies in the field of view of a subject, when the subject wears the device; the method comprising:
identifying on said at least one lens a correction region,
   wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
   wherein said correction region has a two-dimensional double arc shape including a proximal arc and a distal arc, each of said proximal arc and distal arc face said lens center,
   wherein the proximal arc extends between a first proximal end and a second proximal end, and said distal arc extends between a first distal end and a second distal end,
   wherein the first proximal end and said first distal are connected by a first transverse line, and said second proximal end and second distal end is connected by a second transverse line,
   wherein said proximal arc has a proximal distance from said lens center that is between 4 mm and about 6 mm and said distal arc has a distal distance from said lens center that is between about 13 mm and 14 mm; and
   wherein a radial line connecting said first distal end to said lens center defines a first angle that is between 25° and 55° and a radial line connecting said second distal end to said lens center defines a second angle that is between 125° and 155°; and
selecting said location of said correction coordinate in said correction region for placing thereon a correcting element for improving attention performance in a subject having a pre-existing attention disorder and/or for treating a subject having a pre-existing attention disorder.

In accordance with a third aspect of the presently disclosed subject matter there is provided a method of converting an eyeglass into a device for improving performance of subject having a pre-existing attention disorder, and/or for treating a subject having an attention disorder, the method comprising:
selecting a location of at least one correction coordinate on at least one lens of said eyeglass, for placing thereon a correcting element, said selecting comprises at least the steps of
   identifying on said at least one lens a correction region,
      wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
      wherein said correction region has a two-dimensional double arc shape including a proximal arc and a distal arc, each of said proximal arc and distal arc face said lens center,
      wherein the proximal arc extends between a first proximal end and a second proximal end, and said distal arc extends between a first distal end and a second distal end;
      wherein the first proximal end and said first distal are connected by a first transverse line, and said second proximal end and second distal end is connected by a second transverse line;
      wherein said proximal arc has a proximal distance from said lens center that is between 4 mm and about 6 mm and said distal arc has a distal distance from said lens center that is between about 13 mm and 14 mm; and
      wherein a radial line connecting said first distal end to said lens center defines a first angle that is between 25° and 55° and a radial line connecting said second distal end to said lens center defines a second angle that is between 125° and 155°; and
   selecting said location of said correction coordinate in said correction region, the correction coordinate being within the subject's field of view when the eyeglass is worn by the subject; and
placing said at least one correcting element in the selected location in said correction coordinate.

It is to be noted that the second aspect as well as the third aspect of the presently disclosed subject matter do not involve intervention with a subject's body and thus are not to be considered in any manner, methods of treatment of a human body.

In the context of the first, second and third aspects of the presently disclosed subject matter, when referring to a device being administered to the subject in need of the treatment, it is to be understood to encompass any physical device that comprises at least one lens, and when worn by the subject, the at least part of the at least one lens is within the subject's field of view (FOV).

It is to be understood that in the context of the first, second and third aspects of the presently disclosed subject matter, a subject's “visual field” or “field of view” or “FOV” denotes the full angular extent of the area visible to an eye that is fixating straight ahead at any given moment. Thus, in the context of the presently disclosed subject matter, the correction coordinate and the correction region will always be within a subject's theoretical visible area.

Further, in the context of the first, second and third aspects of the presently disclosed subject matter, when referring to a “lens”, it is to be understood to refer to any piece of glass, plastic (e.g. polycarbonate) or other essentially transparent substance, and not necessarily having the functionality of concentration or dispersing light. In other words, the term lens(es), in the context of the presently disclosed subject matter, should not be limited to optical lens(es).

In some examples first, second and third aspects of the presently disclosed subject matter, the device has optical lenses.

In some examples of the presently disclosed subject matter, the device is a Virtual Reality (VR) device or an Augmented Reality (AR) device, including a left side lens and a right-side lens.

In this case, the augmentation element is not physically disposed on lenses, but the image seen on the display of the AR and VR includes an augmenting element position in such location that is equivalent to the augmentation element on the left and/or right side lens.

If an augmenting element exists in the VR or AR device, and a subject is looking into the virtual or augmented world, the device is configured to project the augmenting element into the retina into a specific location, such that the augmenting element is digitally created on the screen so the projection into the retina in a manner/location that is equivalent to the augmenting elements place on lens, if lenses were used instead. The visual image can also be generated on a screen viewed by the subject wearing the device or by a retinal projection system such as a virtual retinal display.

Notably, in VR or AR devices, if the display is not in a single plane (such as holograms, varifocal lenses or alike), the augmenting element is dynamically adjusted to comply with the requirement of simulating the augmenting element being on the lenses.

In some examples of the presently disclosed first, second and third aspects, the device is selected from eyeglasses, sunglasses, zero glasses, pince-nez (eyeglasses without the earpiece), monocle, eye wear viewers or other transparent, partially transparent or regular display on glasses such as LCD, OLED glasses, virtual, augmented or mixed reality glasses, headsets, wearables, binoculars, night vision systems, retinal projection systems, smart glasses (electronic glasses) and the like. In some examples of the presently disclosed first, second and third aspects, the device comprises electronic glasses (also known as smart glasses).

In some examples of the presently disclosed first, second and third aspects, the device is eyeglasses where the lens may be optical or zero lens.

In line with the above, it is to be understood that when referring to a left-side and/or right-side lenses, it encompasses lenses according to their regular meaning (as in eyeglasses) and yet also left-side or right-side portions of a single screen.

In accordance with the first, second and third aspects of the presently disclosed subject matter, the device comprises, located (embedded, adhered, or otherwise placed) on at least one lens, at least one correcting element.

The correcting element can be, in accordance with any of the first, second and third aspects of the presently disclosed subject matter, any type of a mark placed at the correction coordinate. The mark may be in the form of any one of sticker, etch, color, engravement, digital mark, electronic mark, opaque color, projection or the like, each representing an independent embodiment placed or displayed on the correction coordinate within the correction region being in the subject's FOV when the device is worn by the subject.

The correcting element, according to the presently disclosed first, second and third aspects, can be of variable shapes, size, material, texture, dimension, color and/or contour.

The correcting element may have a defined geometrical shape.

The correcting element can be polygon and/or have a curved shape.

The correcting element can be symmetrical or unsymmetrical.

The correcting element can have an irregular shape.

The correcting element can be defined by its dimensions.

In some examples of the presently disclosed first, second and third aspects, the correcting element is defined by any axis or radii having a dimension of between about 1 mm and about 7 mm; at times, between 1 mm and about 6 mm; at times, between 1 mm and about 5 mm; at times, between 1 mm and about 4 mm; at times between 1 mm and about 3 mm; at times, between about 1 mm and 2 mm; at times, between about 2 mm and 7 mm; at times, between about 3 mm and 7 mm; at times between about 4 mm and 7 mm; at times between about 5 mm and 7 mm.

In some examples, the correcting element has at least one dimension of at least about 1.2 mm (but not more than 7 mm); or at least about 1.4 mm; or at least about 1.6 mm; or at least about 1.8 mm; or at least about 2 mm; or at least about 2.2 mm; or at least about 2.4 mm; or at least about 2.6 mm; or at least about 2.8 mm; or at least about 3.0 mm; or at least about 3.2 mm; or at least about 3.4 mm; or at least about 3.6 mm; or at least about 3.8 mm; or at least about 4.0 mm; or at least about 4.2 mm; or at least about 4.4 mm; or at least about 4.6 mm; or at least about 4.8 mm; or at least about 5.0 mm; or at least about 5.2 mm; or at least about 5.4 mm; or at least about 5.6 mm; or at least about 5.8 mm; or at least about 6.0 mm; or at least about 6.2 mm; or at least about 6.4 mm; or at least about 6.6 mm; or at least about 6.8 mm.

In some examples of the presently disclosed first, second and third aspects, the correcting element has a polygonal shape, e.g. of a square or rectangle and the dimension of the correcting element is defined by its longest diagonal being within a range of 1.4 mm and 11 mm.

The device can carry, in accordance with the presently disclosed first, second and third aspects, placed at the correction coordinate, more than one correcting element. If more that one is used, the two or more correcting elements do not necessarily have the same shape, size, material, texture, dimension, color and/or contour.

When more than one correcting element is present on the lens(es), the correcting elements can at least partially overlap.

In accordance with the presently disclosed first, second and third aspects, the correcting element is placed in the presently disclosed correction region.

In accordance with the presently disclosed first, second and third aspects, the correction region is a two (2) dimensional structure defined on the surface of each lens.

The location of the correcting element at the correction coordinate, that is within the correction region are each defined by a polar coordinate system on the surface of the lens. In the context of the first, second and third aspects of the presently disclosed subject matter, the polar coordinate system is a two-dimensional coordinate system in which a position of a point (i) is defined by two values: the radial distance from the lens center (OC) of the lens (known as the radius (rᵢ)), and the angle (Øᵢ) that the radial line connecting the point (i) to the center of the lens makes with a reference axis (known as the angle or azimuth).

In accordance with the presently disclosed first, second and third aspects, both the correction coordinate, and the correction region are defined by the polar coordinate system and share the same lens center. While the correction coordinate is defined by the specific position of a point (i), the correction region defines a segment with boundaries, each point along or within the boundaries can be defined as a point (i) having a radial distance from the lens center and an angle.

In accordance with the presently disclosed first, second and third aspects, the correction coordinate can be any point along the boundaries and any point within the boundaries.

In the context of the first, second and third aspects of the presently disclosed subject matter, the meaning of lens center on the lens (OC) should be understood to correlate with the center of the pupil when the eye is fixating straight ahead, the angular coordinate (Øᵢ) denotes the positive or anticlockwise angle required to reach the point from the 0° ray, defined by the horizontal line or the polar axis (equivalent to a positive X-axis in a Cartesian coordinate plane).

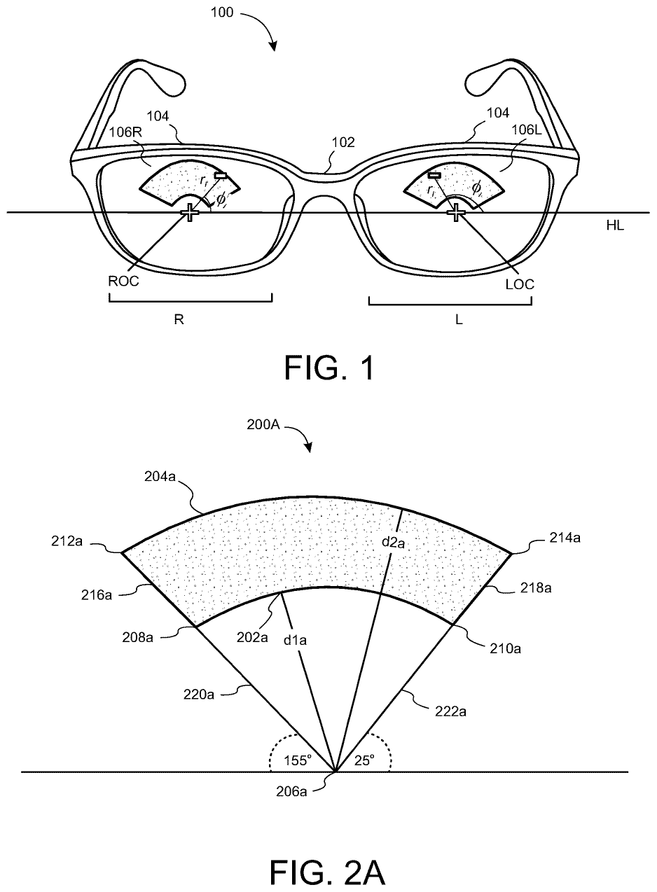

For illustrating the polar coordinate system, reference is made to FIG. 1 providing an illustration of eyeglasses to be worn by the subject, and a simplified presentation of a polar coordinate system. Specifically, FIG. 1 schematically illustrates the construction of a right (R) and left (L) polar coordinate system on a device (100), illustrated in accordance with one example of the presently disclosed subject matter, as eyeglasses.

Device 100 comprises a left eyepiece (L) and a right eyepiece (R) and an arch 102 connecting there between. Each eyepiece consists of a frame 104 and a left lens 106L and right lens 106R.

In addition, each eyepiece has a respective left lens center (LOC) and a right lens center (ROC).

Each of the lens centers, LOC and ROC, constitutes, respectively, the origin of the respective left (L) and right (R) coordinate system, i.e. from which an angular coordinate \acute{Ø}ᵢ and a transverse coordinate (distance) ri are measured. The LOC and ROC are connected by the horizontal line HL.

The device comprises at least one correcting element placed at a correction coordinate within the presently disclosed correction region on at least one lens of the device.

In the context of the first, second and third aspects of the presently disclosed subject matter, the correction region has boundaries defined by an imaginary two-dimensional shape, in the form of at least one double arc shape. In this context, it is to be understood that the double arc shape is not physically marked on the lens(es) of the device and is rather an imaginary two-dimensional shape having defined boundaries, as further detailed below.

In the context of the first, second and third aspects of the presently disclosed subject matter, when referring to a “double arc shape” it is to be understood to mean a segment on the lens enclosed within two discrete arc lines, having their endpoints connected by respective, two transverse lines.

Each of the arcs have a curvature that faces the lens center of polar coordinate system of each lens.

The double arc shape comprises a proximal arc and a distal arc. The proximal arc in the double arc shape has a distance that is greater than the distance of the proximal arc, the distance being measured at the corresponding endpoints of the arcs (the endpoints being referred to by the terms “proximal end”, “distal end”).

In accordance with the first, second and third aspects of the presently disclosed subject matter the proximal arc extends between a first proximal end and a second proximal end, and the distal arc extends between a first distal end and a second distal end.

In accordance with the first, second and third aspects of the presently disclosed subject matter the first proximal end and the first distal end are connected by a first transverse line, and the second proximal end and second distal end are connected by a second transverse line.

The connection of the two arcs by the transverse lines define a close area or segment referred to herein as the correction region.

In the context of the first, second and third aspects of the presently disclosed subject matter, when referring to “transverse line” it is to be understood to encompass a line per se. In some examples of the presently disclosed first, second and third aspects, the transverse line also encompasses a point of connection between the two endpoints of two arcs.

In some examples of the presently disclosed first, second and third aspects, the transverse line is a two-dimensional line with a length.

In some examples of the presently disclosed subject matter, the proximal arc and the distal arc have the same curvature.

In some examples of the first, second and third aspects of the presently disclosed subject matter, the proximal arc and the distal arc are parallelly facing the lens center, i.e. they two arcs in the double arc structure that have the same curvature, are parallel and both face the lens center of the lens.

The distance of an arc from the lens center is determined by extending a radial line from any point along the measured arc towards the optical arc. In other words, the distance is defined by a “radial line” connecting point on the arc and the lens center.

In some examples of the first, second and third aspects of the presently disclosed subject matter, the distance of an arc is provided by a radial line that is extended from the endpoint of the arc. For example, the distance of a proximal arc is determined by connecting the first proximal end or the second proximal end to the lens center, the connecting line being the radial line defining the distance of the respective first proximal end and second proximal end to the lens center.

In some examples of the first, second and third aspects of the presently disclosed subject matter, the distance of the distal arc is determined by connecting the first distal end or the second distal end to the lens center, the connecting line being the radial line defining the distance of the respective first distal end and second distal end to the lens center.

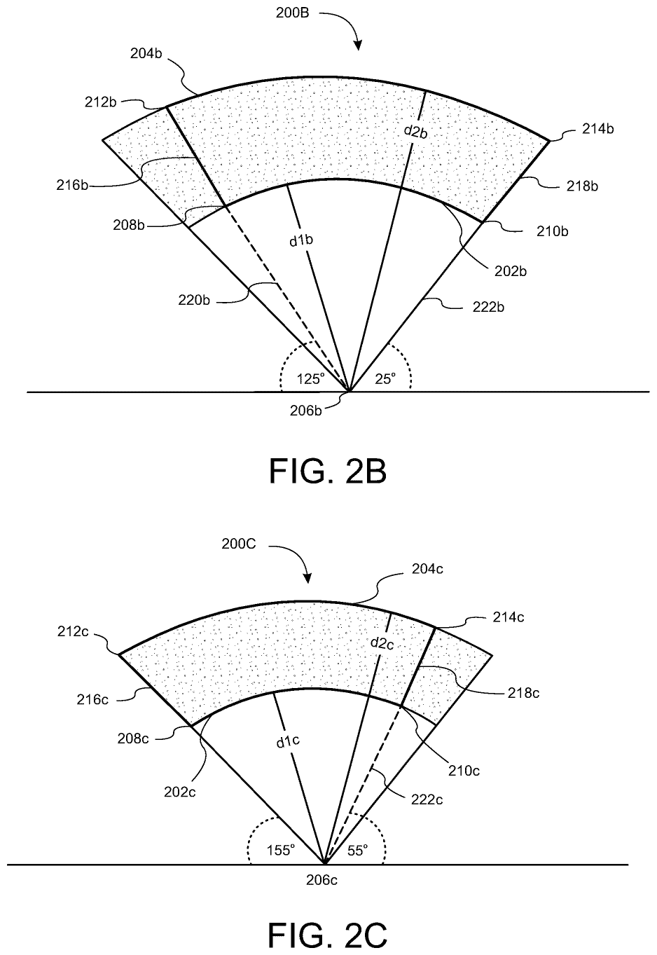

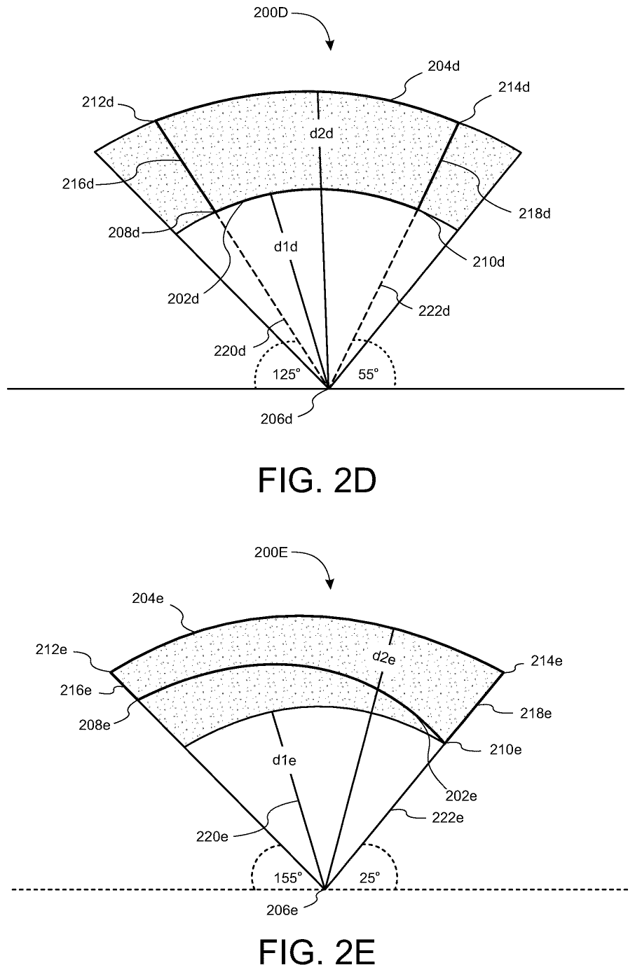

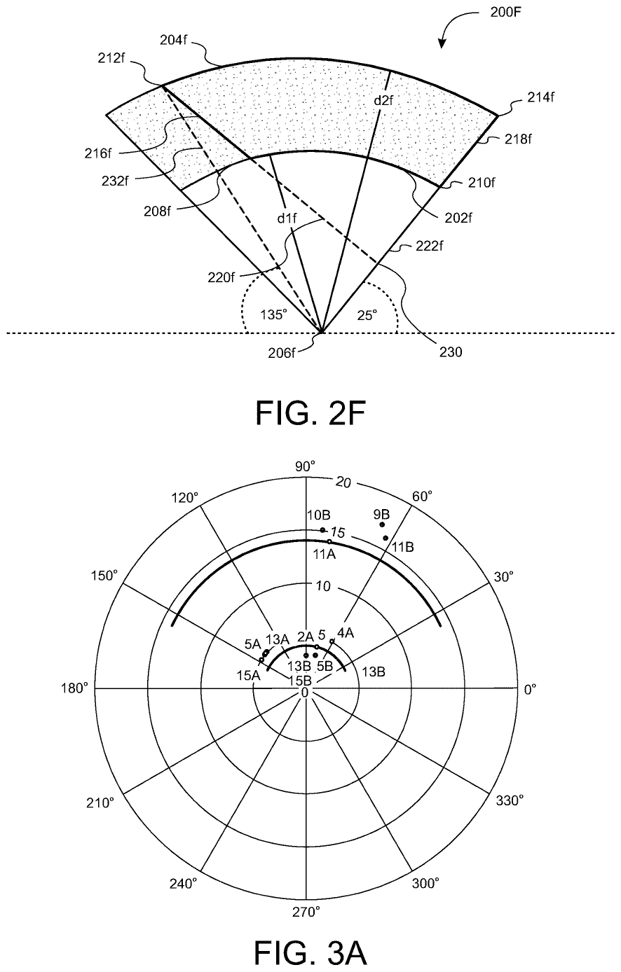

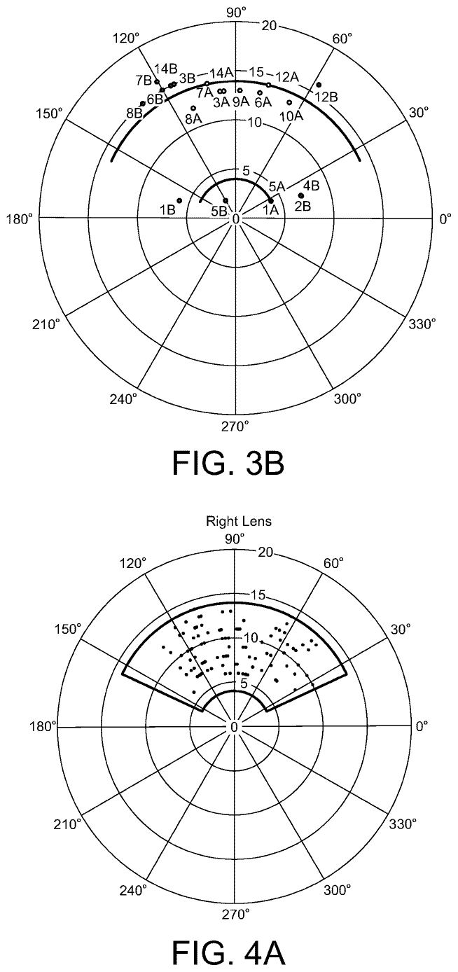

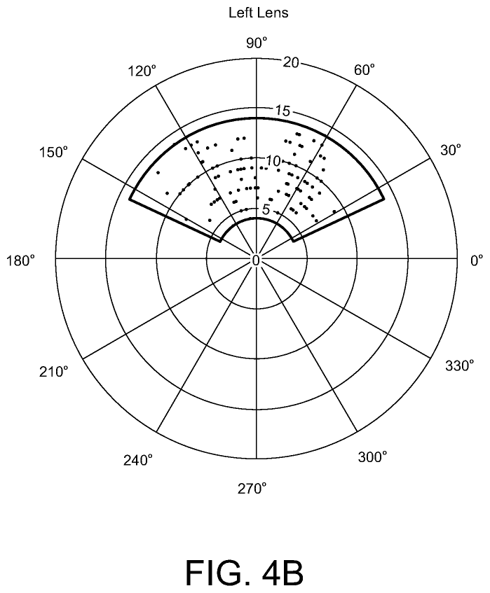

In some examples of the first, second and third aspects of the presently disclosed subject matter, the distance of each of the proximal arc and/or the distal arc from the lens center can be fixed, i.e. the same radial distance from the lens center, along the entire length of the respective arc (as schematically illustrated in any one of FIGS. 2A-2D, 2F without being limited thereto), or can have variable dimensions along the length of the respective arc (as schematically illustrated in FIG. 2E without being limited thereto).

In some examples of the first, second and third aspects of the presently disclosed subject matter of the presently disclosed subject matter, at least at one point along the proximal arc has a proximal distance of 4 mm.

In some examples of the first, second and third aspects of the presently disclosed subject matter of the presently disclosed subject matter, the proximal arc has a fixed proximal distance along the entire length of the proximal arc.

In some examples of the first, second and third aspects of the presently disclosed subject matter of the presently disclosed subject matter, the proximal arc has a fixed proximal distance of 4 mm along the entire length of the proximal arc (i.e. the distance is fixed at 4 mm along the proximal arc).

In some examples of the first, second and third aspects of the presently disclosed subject matter of the presently disclosed subject matter, at least at one point along the proximal arc has a proximal distance of between 4 mm and 6 mm.

In some examples of the first, second and third aspects of the presently disclosed subject matter of the presently disclosed subject matter, the proximal arc has a fixed proximal distance along the entire length of the proximal arc.

In some examples of the first, second and third aspects of the presently disclosed subject matter, the proximal arc has a fixed proximal distance of between 4 mm and 6 mm along the entire length of the proximal arc (i.e. the distance is fixed at 4 mm-6 mm along the proximal arc).

In some examples of the first, second and third aspects of the presently disclosed subject matter, the correction region has boundaries defined by a proximal distance for at least one point along the proximal arc, preferably along each point on the proximal arc, of about 4.1 mm; at times, about 4.2 mm; at times, about 4.3 mm; at times, about 4.4 mm; at times, about 4.5 mm; at times, about 4.6 mm; at times, about 4.7 mm; at times, about 4.8 mm; at times, about 4.9 mm; at times, about 5.0 m; at times, about 5.1 mm; at times, about 5.2 mm; at times, about 5.3 mm; at times, about 5.4 mm; at times, about 5.5 mm; at times, about 5.6 mm; at times, about 5.7 mm; at times, about 5.8 mm; at times, about 5.9 mm; at times, about 6.0 mm, along the proximal arc.

In some examples of the first, second and third aspects of the presently disclosed subject matter, the correction region has boundaries defined by an essentially same distal distance along the entire length of the distal arc.

In some examples of the first, second and third aspects of the presently disclosed subject matter, at least one point along the distal arc has a distal distance of 14 mm.

In some examples of the first, second and third aspects of the presently disclosed subject matter, the distal arc has a fixed distal distance along the entire length of the distal arc.

In some examples of the first, second and third aspects of the presently disclosed subject matter, the distal arc has a fixed distal distance of 14 mm along the entire length of the distal arc (i.e. the distance is fixed at 14 mm along the distal arc).

In some examples of the first, second and third aspects of the presently disclosed subject matter, the distal arc has a distal distance of between 13 mm and 14 mm at least at one point along the distal arc.

In some examples of the first, second and third aspects of the presently disclosed subject matter, the distal arc has a fixed distal distance of between 11 mm and 14 mm along the entire length of the distal arc, (i.e., the distance is fixed at 11 mm-14 mm along the distal arc).

In some examples of the first, second and third aspects of the presently disclosed subject matter, the correction region has boundaries defined by a distal distance for at least one point on along the distal arc, preferably along each point on the distal arc, of about 13.9 mm; at times, about 13.8 mm; at times, about 13.7 mm; at times, about 13.6 mm; at times, about 13.5 mm; at times, about 13.4 mm; at times, about 13.3 mm; at times, about 13.2 mm; at times, about 13.1 mm; at times, about 13.0 mm; at times, about 12.9; at times about 12.8 mm; at times, about 12.7 mm; at times, about 12.6 mm; at times, about 12.5 mm; at times, about 12.4 mm; at times, about 12.3 mm; at times, about 12.2 mm; at times, about 12.1 mm; at times, 11.0 mm.

In some examples of the first, second and third aspects of the presently disclosed subject matter, at least one of the proximal arc and the distal arc have a variable distance from the lens center, as illustrated in FIG. 2E.

In some examples of the first, second and third aspects of the presently disclosed subject matter, the first angle of the double arc shape, that is defined by extending a radial line from the first distal end towards the lens center is equal to or greater than 25°.

In some examples of the first, second and third aspects of the presently disclosed subject matter, the first angle of the double arc shape is between 25° and 55°; at times between 25° and 50°; at times; between 25° and 45°; at times between 25° and 40°; at times, between 25° and 35°.

In some examples of the first, second and third aspects of the presently disclosed subject matter, the second angle of the double arc shape, that is defined by extending a radial line from the second distal end towards the lens center is equal to or smaller than 155°.

In some examples of the first, second and third aspects of the presently disclosed subject matter, the second angle of the double arc shape is between 155° and 125°; at times between 155° and 130°; at times; between 155° and 135°; at times between 155° and 140°; at times, between 155° and 145°.

In some examples of the first, second and third aspects of the presently disclosed subject matter, the proximal arc has a fixed proximal distance of 4 mm along an entire length of the proximal arc and the distal arc has a fixed distal distance of 14 mm along an entire length of the distal arc.

In some examples of the first, second and third aspects of the presently disclosed subject matter the first angle is 25° and the second angle is 155.

In some examples of the first, second and third aspects of the presently disclosed subject matter, the proximal arc has a fixed proximal distance of 4 mm along an entire length of the proximal arc and a first angle of 25° and the distal arc has a fixed distal distance of 14 mm along an entire length of the distal arc and a second angle of 155°, as schematically illustrated in FIG. 2A.

In some examples of the first, second and third aspects of the presently disclosed subject matter, the proximal arc has a fixed proximal distance of 4 mm along an entire length of the proximal arc and a first angle of 25° and the distal arc has a fixed distal distance of 14 mm along an entire length of the distal arc and a second angle smaller than 155°, e.g. 125° as schematically illustrated in FIG. 2B.

In some examples of the first, second and third aspects of the presently disclosed subject matter, the proximal arc has a fixed proximal distance of 4 mm along an entire length of the proximal arc and a first angle that is greater than 25°, e.g. 55° and the distal arc has a fixed distal distance of 14 mm along an entire length of the distal arc and a second angle of 155°, as schematically illustrated in FIG. 2C.

In some examples of the first, second and third aspects of the presently disclosed subject matter, the proximal arc has a fixed proximal distance of 4 mm along an entire length of the proximal arc and a first angle greater than 25°, e.g. 55° and the distal arc has a fixed distal distance of 14 mm along an entire length of the distal arc and a second angle that is smaller than 155°, e.g. 125°, as schematically illustrated in FIG. 2D.

In some examples of the first, second and third aspects of the presently disclosed subject matter, at least one of the proximal arc and the distal arc has a variable distance from said lens center, as schematically illustrated in FIG. 2E.

In some examples of first, second and third aspects of the presently disclosed subject matter, the radial line connecting any one of the first distal end and second distal end is at an angle that is different from an angle defined by a proximal radial line connecting a respective first proximal end or second proximal end. This being schematically illustrated in FIG. 2F.

In some examples of the first, second and third aspects of the presently disclosed subject matter, the radial line connecting the first distal end with the lens center converges with the first transverse line.

In some examples of the first, second and third aspects of the presently disclosed subject matter, the radial line connecting the second distal end with the lens center converges with the second transverse line.

In some examples of the first, second and third aspects of the presently disclosed subject matter, the radial line connecting the first distal end of a double arc shape, with the lens center, converges with the first transverse line. In other words, the first radial line and the first transverse line overlap.

Similarly, in some examples of the first, second and third aspects of the presently disclosed subject matter, the radial line connecting the second distal end of a double arc shape, with the lens center, converges with the second transverse line. In other words, the second radial line and the second transverse line overlap.

Such examples, where the radial lines and the transverse lines overlap, are illustrated in any one of FIGS. 2A-2E, without being limited thereto.

As noted hereinabove and below, the device comprises a left lens and/or a right lens. Correcting element(s) can be applied on the left lens, on the right lens or on both, as long as the correcting element is placed at a correction coordinate that is within the correction region as disclosed herein.

In some examples of the first aspect of the presently disclosed subject matter, the method comprises administering a device that holds at least one first correcting element in a left correction region of the left lens, and at least one second correcting element in a right correction region of the right lens.

In some examples of first aspect of the presently disclosed subject matter, the method comprises administering a device that holds two or more correcting elements in the correction region of at least one lens.

In some examples of the first aspect of the presently disclosed subject matter, when the device holds two or more correcting element on a lens, the two or more correcting elements can at least partially overlap.

In some examples of the second and third aspect of the presently disclosed subject matter, the selection of the location of the at least one correction coordinate for placing thereon a correcting element comprises a priori identification of a correction region.

In the context of the second and third aspects of the presently disclosed subject matter, the identification of the correction region comprises at least identification of the lens center and calculating therefrom the boundaries of the correction region (the radial distances of the arcs and their angle at their respective first and second ends).

In some examples of the second and third aspects of the presently disclosed subject matter, the selection of at least one correction coordinate within the correction region comprises:
assessing attention performance of a subject;
determining boundaries of the correction region
selecting a correction coordinate within said correction region, according to the subject's performance.

In the context of the second and third aspects of the presently disclosed subject matter, said “assessing” is based on the subject's performance during a task in an acceptable test for assessing attention, as further elaborated hereinbelow.

In some examples of the presently disclosed first, second and third aspects, the determining of boundaries of the correction region is based on the following:
defining a polar coordinate system in the at least one lens; the polar coordinate system measured from a lens center of the at least one lens (the lens center overlapping the coordinate system center), wherein the lens center is configured to be horizontally aligned with an optical center of an eye (left or right) of the subject, when the device is worn by the subject;
determining boundaries of the left side correction region and/or right-side correction region in the at least one lens, each of the left side correction region and right-side correction region having independently the two dimensional double arc shape as defined herein wherein at least one point on the proximal arc of the double arc shape has a proximal distance from said lens center that is between 4 mm and about 6 mm and at least one point on the distal arc has a distal distance from said lens center that is between about 13 mm and 14 mm; and the radial line connecting the first distal end to the lens center defines a first angle that is between 25° and 55° and a radial line connecting the second distal end to the lens center defines a second angle that is between 125° and 155°.

In some examples of the presently disclosed first, second and third aspects, the location of the selected correction coordinate is within the correction region and in accordance with the subject's performance in the test task. This location would then be the location of placement of the correcting element for attention performance of a subject when the device is worn by the subject.

In some examples of the first, second and third aspects of the presently disclosed subject matter, the location of the correction coordinate within the correction region and accordingly the location of the correcting element to be placed thereat is defined by an angle Ø, and a distance r of a radial line connecting the center of placement of the correcting element and the lens center.

In some examples of the first, second and third aspects of the presently disclosed subject matter, the correcting element is placed or to be placed at an angle of Ø=26°, and a distance r=4 on a left lens.

In some examples of the first, second and third aspects of the presently disclosed subject matter, the correcting element is at an angle of Ø=75°, and a distance r=4 on a right lens.

In some examples of the first, second and third aspects of the presently disclosed subject matter, the correcting element is placed or to be placed at an angle of Ø=95°, and a distance r=13 on a left lens.

In some examples of the first, second and third aspects of the presently disclosed subject matter, the correcting element is placed or to be placed at an angle of Ø=61°, and a distance r=5 on a right lens.

In some examples of the first, second and third aspects of the presently disclosed subject matter, a first correcting element is placed or to be placed at an angle of Ø=141°, and a distance r=5 on a right lens and at an angle of Ø=26° and at distance of r=4 on the left lens.

In some examples of the first, second and third aspects of the presently disclosed subject matter, the correcting element is placed or to be placed at an angle of Ø=79° and at distance of r=13 on the left lens.

In some examples of the first, second and third aspects of the presently disclosed subject matter, the correcting element is placed or to be placed at an angle of Ø=102° and at distance of r=14 on the left lens.

In some examples of the first, second and third aspects of the presently disclosed subject matter, the correcting element is placed or to be placed at an angle of Ø=111° and at distance of r=12 on the left lens.

In some examples of the first, second and third aspects of the presently disclosed subject matter, the correcting element is placed or to be placed at an angle of Ø=88° and at distance of r=13 on the left lens.

In some examples of the first, second and third aspects of the presently disclosed subject matter, the correcting element is placed or to be placed at an angle of Ø=65° and at distance of r=13 on the left lens.

In some examples of the first, second and third aspects of the presently disclosed subject matter, the correcting element is placed or to be placed at an angle of Ø−81° and at a distance of r=14 on the right lens.

In some examples of the first, second and third aspects of the presently disclosed subject matter, the correcting element is placed or to be placed at an angle of Ø=76° and at a distance of r=14 on the left lens.

In some examples of the first, second or third aspects of the presently disclosed subject matter, the location of the correction coordinate for placing there the correcting element is as presented in Table 1:

TABLE 1
Location of correction coordinates with the presently disclosed
correction region for improving and/or treating ADHD

| | Right Lens | | Left lens | |
|---|---|---|---|---|
| Correction | r | | r | |
| Coordinate No. | (mm) | ∅° | (mm) | ∅° |
| 1 | 6 | 95 | 9 | 31 |
| 2 | 7 | 122 | 7 | 32 |
| 3 | 7 | 116 | 7 | 37 |
| 4 | 7 | 124 | 7 | 45 |
| 5 | 7 | 124 | 9 | 46 |
| 6 | 7 | 127 | 7 | 47 |
| 7 | 8 | 115 | 8 | 47 |
| 8 | 6 | 140 | 6 | 49 |
| 9 | 7 | 124 | 8 | 49 |
| 10 | 10 | 96 | 10 | 50 |
| 11 | 7 | 123 | 7 | 51 |
| 12 | 8 | 116 | 9 | 52 |
| 13 | 8 | 133 | 6 | 53 |
| 14 | 13 | 107 | 11 | 53 |
| 15 | 8 | 117 | 10 | 53 |
| 16 | 8 | 128 | 6 | 53 |
| 17 | 8 | 125 | 6 | 55 |
| 18 | 12 | 108 | 9 | 56 |
| 19 | 11 | 106 | 12 | 56 |
| 20 | 10 | 112 | 8 | 57 |
| 21 | 11 | 98 | 12 | 58 |
| 22 | 9 | 120 | 9 | 58 |
| 23 | 8 | 115 | 8 | 59 |
| 24 | 12 | 108 | 13 | 60 |
| 25 | 12 | 119 | 8 | 60 |
| 26 | 7 | 112 | 11 | 61 |
| 27 | 11 | 99 | 14 | 61 |
| 28 | 10 | 119 | 9 | 63 |
| 29 | 10 | 120 | 9 | 63 |
| 30 | 11 | 131 | 7 | 63 |
| 31 | 10 | 110 | 13 | 64 |
| 32 | 8 | 59 | 7 | 65 |
| 33 | 6 | 78 | 5 | 65 |
| 34 | 9 | 133 | 8 | 67 |
| 35 | 10 | 110 | 10 | 67 |
| 36 | 9 | 124 | 9 | 67 |
| 37 | 10 | 122 | 8 | 68 |
| 38 | 12 | 112 | 12 | 68 |
| 39 | 14 | 109 | 12 | 69 |
| 40 | 12 | 121 | 9 | 69 |
| 41 | 12 | 111 | 11 | 70 |
| 42 | 11 | 115 | 10 | 71 |
| 43 | 12 | 121 | 13 | 71 |
| 44 | 13 | 108 | 13 | 72 |
| 45 | 10 | 127 | 7 | 72 |
| 46 | 13 | 108 | 13 | 72 |
| 47 | 14 | 111 | 12 | 74 |
| 48 | 12 | 121 | 10 | 75 |
| 49 | 13 | 121 | 10 | 75 |
| 50 | 13 | 115 | 12 | 76 |
| 51 | 10 | 72 | 9 | 76 |
| 52 | 7 | 64 | 6 | 76 |
| 53 | 7 | 73 | 6 | 77 |
| 54 | 12 | 132 | 9 | 84 |
| 55 | 11 | 88 | 9 | 87 |
| 56 | 6 | 81 | 6 | 89 |
| 57 | 8 | 96 | 7 | 89 |
| 58 | 8 | 78 | 7 | 91 |
| 59 | 9 | 84 | 8 | 91 |
| 60 | 12 | 85 | 10 | 93 |
| 61 | 7 | 89 | 9 | 93 |
| 62 | 7 | 88 | 7 | 95 |
| 63 | 13 | 92 | 12 | 96 |
| 64 | 10 | 98 | 9 | 98 |
| 65 | 8 | 97 | 7 | 98 |
| 66 | 13 | 96 | 10 | 99 |
| 67 | 11 | 89 | 12 | 99 |
| 68 | 5 | 79 | 4 | 99 |
| 69 | 11 | 87 | 9 | 100 |
| 70 | 10 | 94 | 8 | 101 |
| 71 | 6 | 86 | 5 | 102 |
| 72 | 9 | 94 | 9 | 102 |
| 73 | 13 | 45 | 11 | 103 |
| 74 | 4 | 95 | 7 | 105 |
| 75 | 13 | 48 | 9 | 107 |
| 76 | 10 | 28 | 5 | 108 |
| 77 | 13 | 63 | 12 | 110 |
| 78 | 8 | 99 | 6 | 110 |
| 79 | 12 | 50 | 7 | 112 |
| 80 | 7 | 101 | 6 | 112 |
| 81 | 11 | 112 | 10 | 113 |
| 82 | 9 | 49 | 12 | 114 |
| 83 | 8 | 104 | 9 | 115 |
| 84 | 10 | 44 | 10 | 116 |
| 85 | 11 | 51 | 9 | 117 |
| 86 | 13 | 63 | 13 | 117 |
| 87 | 11 | 53 | 9 | 117 |
| 88 | 10 | 56 | 11 | 119 |
| 89 | 12 | 65 | 12 | 119 |
| 90 | 11 | 68 | 13 | 120 |
| 91 | 14 | 67 | 11 | 120 |
| 92 | 11 | 57 | 8 | 121 |
| 93 | 9 | 41 | 7 | 122 |
| 94 | 12 | 62 | 13 | 123 |
| 95 | 11 | 51 | 7 | 124 |
| 96 | 12 | 67 | 14 | 126 |
| 97 | 6 | 100 | 4 | 126 |
| 98 | 11 | 57 | 10 | 126 |
| 99 | 12 | 46 | 7 | 127 |
| 100 | 10 | 35 | 7 | 130 |
| 101 | 11 | 62 | 10 | 132 |
| 102 | 8 | 73 | 10 | 133 |
| 103 | 9 | 66 | 10 | 137 |
| 104 | 9 | 82 | 13 | 139 |
| 105 | 9 | 63 | 10 | 140 |
| 106 | 8 | 31 | 6 | 141 |
| 107 | 7 | 48 | 6 | 141 |
| 108 | 6 | 76 | 11 | 144 |
| 109 | 7 | 47 | 8 | 151 |

It is to be noted that in accordance with the first, second and third aspects of the presently disclosed subject matter, each pair of radial line (r) and angle (Ø) of a left lens and/or a right lens in Table 1 represent an independent correction coordinate of the presently disclosed subject matter. Similarly, each pair of correction coordinates from the left lens and right lens (identified by an arbitrary correction coordinate No.) represent independent pairs of correction coordinates of the presently disclosed subject matter.

Each independent correction coordinates and/or pair of correction coordinates are suitable for performing the method according to the first aspect of the presently disclosed subject matter.

Further, each independent correction coordinates and/or pair of correction coordinates are suitable for performing the method according to the second aspect of the presently disclosed subject matter.

The presently disclosed first, second and third aspects provide methods utilized for treating or at least improving attention performance in a subject having attention disorder.

In the context of the first, second and third aspects of the presently disclosed subject matter, by the term “attention disorder” it is meant to encompass any condition in which a subject may have a subjective or objective difficulty in any one of paying attention, staying focused, concentrating on tasks, following instructions, organizing tasks, completing tasks, managing time, planning, coping with stress, remaining seated, engaging in activities quietly, paying close attention to details, reading, controlling emotions, and controlling impulsive behavior (e.g. unable to wait their turn, acting without thinking, interrupting conversations, little or no sense of danger). As appreciated, such disorder can interfere with their ability to perform well in school, work, and social settings.

In some examples of the first, second and third aspects of the presently disclosed subject matter, the subject having an attention disorder, is one which has been diagnosed with a pre-existing attention disorder.

In preferred examples, the attention disorder is Attention Deficit Hyperactivity Disorder (ADHD). In the context of the presently disclosed subject matter, by the term “Attention Deficit Hyperactivity Disorder” or “ADHD” it is meant to a specific type of attention disorder that is characterized by at least one, preferably more than one of the symptoms selected from the group consisting of inattention, hyperactivity, and impulsivity. As appreciated, subjects with ADHD may struggle to follow instructions, complete tasks, stay organized, and manage their time effectively. They may also be easily distracted, forgetful, and prone to making impulsive decisions.

Therefore, it is desirable to provide means for improving performance of subjects with attention disorder, all the more with ADHD. The means include, inter alia, the converting of an eyeglass into a device for improving performance of subject having an attention disorder.

Thus, in some examples of the first, second and third aspects of the presently disclosed subject matter the attention disorder is ADHD.

In the context of the present disclosure, the term “attention performance” is to be understood to include performance of the subject according to at least one attention and/or cognitive and/or physiological parameter.

In some examples of the first, second, and third aspects of the presently disclosed subject matter, the term “treatment” or “treat” or “improving attention performance” should be understood to mean any improvement in a subject's difficulties associated with attention disorder, and specifically, with ADHD. The determination of the improvement can be subjective, e.g. based on feedback received from the subject receiving the treatment, including questioners and rating scales (e.g. ADHD Rating scale, Adult ADHD Self-Report Scale (ASRS), Attention-Deficit/Hyperactivity Disorder Investigator Symptom Rating Scale (AISRS), the Conners rating scale, the Behavior Rating Inventory of Executive Function (BRIEF), Vanderbilt Rating Scale, Brown scales, Child Behavior Checklist, Adult ADHD Clinical Diagnostic Scale (ACDS), Barkley scales), and/or objective, e.g. based on acceptable tests for assessing attention.

In some examples of the first, second, and third aspects of the presently disclosed subject matter, the improvement is assessed by conducting on the subject being treated in accordance with the presently disclosed subject matter, the Continuous Performance Test (CPT), e.g. the Conners' CPT. As appreciated by those versed in the art, CPT is a computerized test that measures a person's attention, impulsivity, and reaction time. It requires the subject to press a button whenever a certain letter or symbol appears on the screen. Other CPT are known, such as Test of Variables of Attention (TOVA), BRC (Brain Resource Cognition), MOXO, Integrated Visual and Auditory (IVA), QbTest, NeuroTrax, Tests of Attentional Performance (TAP).

In some examples of the first, second, and third aspects of the presently disclosed subject matter, the improvement is assessed by conducting on the subject being treated in accordance with the presently disclosed subject matter, the Trial Making Test (TMT). The TMT is a neuropsychological test to assess attention and cognitive flexibility in individuals with ADHD. The test consists of two parts, TMT-A and TMT-B. In TMT-A, the subject is asked to connect a series of numbered circles in ascending order as quickly as possible. In TMT-B, the subject is asked to connect a series of circles alternating between numbers and letters in ascending and alphabetical order, respectively.

In some examples of the first, second, and third aspects of the presently disclosed subject matter, the improvement is assessed by conducting the Stroop test on the subject being treated. The Stroop test is another neuropsychological test to assess attention and inhibition in subjects with ADHD. The Stroop is a neuropsychological test of selective attention and processing speed, which measures the ability to inhibit cognitive interference when two competing stimuli are presented simultaneously. The individual is asked to name the colored box, as fast and accurately as possible. The test includes four conditions: Neutral presentation—where only the ink color is presented, Congruent presentation—the ink color and the printed word are the same, Incongruent presentation —the ink color conflicts with the printed word, and Negative Priming presentation—where the distractive word is the same as the subsequent target ink color.

In some examples of the first, second, and third aspects of the presently disclosed subject matter, the improvement is assessed by conducting any one of or combination of Digit symbol substitution test, Digit Span test, Wechsler Adult Intelligence Scale (WAIS), Wisconsin Card Sorting Test (WCST), Go/No Go test (GNG), Symbol Search, California Verbal Learning Test-II, Tower of London test, Cancellation test, D2, Visual Search and Attention Test (VSAT), Stop Single Task.

In some examples of the first, second, and third aspects of the presently disclosed subject matter, the improvement is determined by a physiological parameter. Without being limited thereto, one or more physiological and/or behavioral sensors may be used to measure physiological and behavioral biomarkers in the subject to a test performed, in a manner know per se. Examples of physiological and behavioral sensors include, but are not limited to blood pressure, dynamometer sensor, respiration monitor belt, stress thermometer, Galvanik skin response, electroocoulogram, eye tracking, electroencephalogram (EEG), electrocardiogram (ECG, EKG), Electromyography (EMG).

The determination of improvement can be based on a single test or a combination of tests acceptable for assessing attention and is typically conducted by a professional practitioner.

Reference is now made to FIGS. 2A-2F which schematically illustrate different dimensions for correction regions according to non-limiting examples of the presently disclosed subject matter,

FIG. 2A schematically illustrates a double arc shape 200A, including a proximal arc 202a and a distal arc 204a, both proximal arc 202a and distal arc 204a facing the lens center 206a. The proximal arc 202a extends between a first proximal end 208a and a second proximal end 210a and said distal arc 204a extends between a first distal end 212a and a second distal end 214a.

First proximal end 208a and first distal end 212a are connected by a first transverse line 216a, while second proximal end 210a and second distal end 214a are connected by a second transverse line 218a. If extended, first transverse line 216a and second transverse line 218a, would meet at lens center 206a, as also illustrated in FIG. 2A by first radial line 220a and second radial line 222a.

Double arc shape 200A, is further defined by an angle α₁ₐ that is defined by first radial line 220a being, as noted above, an extension of transverse line 216a towards lens center 206a, that is equal to 155°, an angle α₂ₐ defined by second radial line 222a, being, as noted above, an extension of transverse line 218a towards lens center 206a, that is equal to 25°.

Double arc shape 200A is further characterized by a fixed distance d₁ₐ of proximal arc 202a from lens center 206a of 4 mm and a fixed distance d₂ₐ of distal arc 204a from lens center 206a of 14 mm.

Reference is now made to FIG. 2B which schematically illustrates another example of 2-dimensional double arc structure 200B defining boundaries of correction region in accordance with another non-limiting example of the presently disclosed subject matter.

For simplicity, like reference numerals to those used in FIG. 2A are used to identify components having a similar function in FIG. 2B, while replacing the letter “a” associated with each reference number with “b” For example, component 206b in FIG. 2B is the same lens center 206a having a similar function in FIG. 2A.

In addition, for the sake comparison between the different non-limiting examples, the boundaries of double arc shape of FIG. 2A is marked in FIG. 2B by dashed lines.

Specifically, FIG. 2B schematically illustrates a double arc shape 200B, including a proximal arc 202b and a distal arc 204b, both proximal art 202b and distal arc 204b facing the lens center 206b. The proximal arc 202b extends between a first proximal end 208b and a second proximal end 210b and distal arc 204b extends between a first distal end 212b and a second distal end 214b.

First proximal end 208b and first distal end 212b are connected by a first transverse line 216b, while second proximal end 210b and second distal end 214b are connected by a second transverse line 218b. If extended, first transverse line 216b and second transverse line 218b, would meet at lens center 206b is illustrated in FIG. 2B by first radial line 220b and second radial line 222b.

Double arc 200B is further characterized by a fixed distance d_{1b} of proximal arc 202b from lens center 206b of 4 mm and a fixed distance d_{2b} of distal arc 204b from lens center 206b of 14 mm.

Double arc shape 200B is further characterized by an angle α_{1b} that is defined by first radial line 220b, being as noted above, an extension of transverse line 216b towards lens center 206b that is smaller than 155° but not less than 125°, and a second angle @2b defined by second radial line 222b, being, as noted above, an extension of transverse line 218b towards lens center 206b, that is equal to 25°.

As a result, the lengths of proximal arc 202b (dimensions between first proximal end 208b and second proximal end 210b) and distal arc 204b (dimensions between first distal end 212b and second distal end 214b) are shorter than the length of proximal arc 202a or distal arc 204a in FIG. 2A.

Reference is now made to FIG. 2C which schematically illustrates another example of 2-dimensional double arc structure 200C defining boundaries of correction region in accordance with another non-limiting example of the presently disclosed subject matter.

For simplicity, like reference numerals to those used in FIG. 2A are used to identify components having a similar function in FIG. 2C, while replacing the letter “a” associated with each reference number with “c” For example, component 206c in FIG. 2C is the same lens center 206a having a similar function in FIG. 2A.

In addition, for the sake comparison between the different non-limiting examples, the boundaries of double arc shape of FIG. 2A is marked in FIG. 2C by dashed lines.

Specifically, FIG. 2C schematically illustrates a double arc shape 200C, including a proximal arc 202c and a distal arc 204c, both proximal art 202c and distal arc 204c facing the lens center 206c. The proximal arc 202c extends between a first proximal end 208c and a second proximal end 210c and distal arc 204c extends between a first distal end 212c and a second distal end 214c.

First proximal end 208c and first distal end 212c are connected by a first transverse line 216c, while second proximal end 210c and second distal end 214c are connected by a second transverse line 218c. If extended, first transverse line 216c and second transverse line 218c, would meet at lens center 206c is illustrated in FIG. 2C by first radial line 220c and second radial line 222c.

Double arc 200C is further characterized by a fixed distance d_{1c} of proximal arc 202c from lens center 206c of 4 mm and a fixed distance d_{2c} of distal arc 204c from lens center 206c of 14 mm.

Double arc shape 200C is further characterized by an angle α_{1c} that is defined by first radial line 220c, being as noted above, an extension of transverse line 216c towards lens center 206c that is equal to 155° and a second angle α_{2c} defined by second radial line 222c, being, as noted above, an extension of transverse line 218c towards lens center 206c, and in this non-limiting example, greater than 25° but not more than 55°.

As a result, the lengths of proximal arc 202c (dimensions between first proximal end 208c and second proximal end 210c) and of distal arc 204c (dimensions between first distal end 212c and second distal end 214c) are shorter than the length of proximal arc 202a or distal arc 204a in FIG. 2A.

Reference is now made to FIG. 2D which schematically illustrates another example of 2-dimensional double arc structure 200D defining boundaries of correction region in accordance with another non-limiting example of the presently disclosed subject matter.

For simplicity, like reference numerals to those used in FIG. 2A are used to identify components having a similar function in FIG. 2D, while replacing the letter “a” associated with each reference number with “d” For example, component 206d in FIG. 2D is the same lens center 206a having a similar function in FIG. 2A.

In addition, for the sake comparison between the different non-limiting examples, the boundaries of double arc shape of FIG. 2A is marked in FIG. 2D by dashed lines.

Specifically, FIG. 2D schematically illustrates a double arc shape 200D, including a proximal arc 202d and a distal arc 204d, both proximal art 202d and distal arc 204d facing the lens center 206d. The proximal arc 202d extends between a first proximal end 208d and a second proximal end 210d and distal arc 204d extends between a first distal end 212d and a second distal end 214d.

First proximal end 208d and first distal end 212d are connected by a first transverse line 216d, while second proximal end 210d and second distal end 214d are connected by a second transverse line 218d. If extended towards lens center 206d, first transverse line 216d and second transverse line 218d, would meet at this lens center 206d, the extensions being illustrated in FIG. 2D by first radial line 220d and second radial line 222d.

Double arc 200D is further characterized by a fixed distance d₁ₐ of proximal arc 202d from lens center 206d of 4 mm and a fixed distance d₂ₐ of distal arc 204d from lens center 206d of 14 mm.

Double arc shape 200D is further characterized by an angle did that is defined by first radial line 220d, being as noted above, an extension of transverse line 216d towards lens center 206d that is smaller than 155° (but greater than) 125° and a second angle α_{2d} defined by second radial line 222d, being, as noted above, an extension of transverse line 218d towards lens center 206d, and in this non-limiting example, greater than 25° but not more than 55°.

As a result, the length of both proximal arc 202d and distal arc 204d are shorter than the length of proximal arc 202a and distal arc 204a in FIG. 2A.

Reference is now made to FIG. 2E which schematically illustrates another example of 2-dimensional double arc structure 200E defining boundaries of correction region in accordance with another non-limiting example of the presently disclosed subject matter.

For simplicity, like reference numerals to those used in FIG. 2A are used to identify components having a similar function in FIG. 2E, while replacing the letter “a” associated with each reference number with “e” For example, component 206e in FIG. 2E is the same lens center 206a having a similar function in FIG. 2A.

In addition, for the sake comparison between the different non-limiting examples, the boundaries of double arc shape of FIG. 2A is marked in FIG. 2E by dashed lines.

Specifically, FIG. 2E schematically illustrates a double arc shape 200E including a proximal arc 202e and a distal arc 204e, both proximal arc 202e and distal arc 204e facing the lens center 206e. Yet, in this non-limiting example, proximal arc 202e and distal arc 204e are not parallel.

Proximal arc 202e extends between a first proximal end 208e and a second proximal end 210e and distal arc 204e extends between a first distal end 212e and a second distal end 214e.

First proximal end 208e and first distal end 212e are connected by a first transverse line 216e, while second proximal end 210e and second distal end 214e are connected by a second transverse line 218e. If extended towards lens center 206e, first transverse line 216e and second transverse line 218e, would meet at the lens center 206e, the extensions being illustrated in FIG. 2F as first radial line 220e and as second radial line 222e.

Double arc 200E is further characterized by a distance d₁ₑ of proximal arc 202e from lens center 206e that equal to 4 mm at second proximal end 210e, however, increases towards first proximal end 208e. Distal arc 204e is characterized by a fixed distance d₂ₑ from lens center 206e that is equal to 14 mm and notwithstanding the increase in distance d_{1f}, d₂ₑ is always greater than die.

Double arc shape is further characterized by an angle die that is defined by first radial line 220e, being as noted above, an extension towards lens center 206e of transverse lines 216e, the angle die being in this example equal to 155°, and angle d₂ₑ defined by second radial line 222e, being an extension towards lens center 206e of transverse lines 218e being in this example 25°.

Reference is now made to FIG. 2F which schematically illustrates another example of 2-dimensional double arc structure 200F defining boundaries of correction region in accordance with another non-limiting example of the presently disclosed subject matter.

For simplicity, like reference numerals to those used in FIG. 2A are used to identify components having a similar function in FIG. 2F, while replacing the letter “a” associated with each reference number with “f”. For example, component 206f in FIG. 2F is the same lens center 206a in FIG. 2A having a similar function.

In addition, for the sake comparison between the different non-limiting examples, the boundaries of double arc shape of FIG. 2A is marked in FIG. 2F by dashed lines.

Specifically, FIG. 2F schematically illustrates a double arc shape 200F, including a proximal arc 202f and a distal arc 204f, both proximal art 202f and distal arc 204f facing the lens center 206f. Proximal arc 202f extends between a first proximal end 208f and a second proximal end 210f and distal arc 204f extends between a first distal end 212f and a second distal end 214f.

First proximal end 208f and first distal end 212f are connected by a first transverse line 216f, while second proximal end 210f and second distal end 214f are connected by a second transverse line 218f.

If extended, first transverse line 216f and second transverse line 218f would intersect at intersection point 230, in other words, different from radial lines 220a and 222a in FIG. 2A. Further, as seen in FIG. 2F radial line 220f has an angle with lens center 206f that is different from an angle formed by a radial line (not shown) connecting proximal end 208f to lens center 206f.

Double arc 200F is further characterized by a fixed distance d_{1f} of proximal arc 202f from lens center 206f of 4 mm and a fixed distance d_{2f} of distal arc 204f from optical center 206f of 14 mm.

Double arc shape 200F is further characterized by an angle gif, defined by radial line 232f taken from first distal end 212f towards lens center 206f, that is, in this non-limiting example, smaller than 155° and an angle α_{2f} defined by second radial line 222f that is, in this non-limiting example 218f towards lens center 206f, and is greater than 25°.

As a result, the length of at least proximal arc 202f (dimensions between first proximal end 208f and second proximal end 210f) is shorter than the length of proximal arc 202a in FIG. 2A.

### EMBODIMENTS

Some non-limiting embodiments encompassed by the present disclosure are defined in the following numbered clauses:

1. A method for treating a subject having a pre-existing, attention disorder, the method comprising administering, to the subject having the attention disorder, a device comprising at least one lens, and at least one correcting element disposed on the at least one lens,
wherein the at least one correcting element is disposed so as to be within the subject's field of view when the device is worn;
wherein the at least one correcting element is placed at a correction coordinate that is within a correction region;
wherein the correction region and the correction coordinate are each defined using a polar coordinate system measured from a lens center of a left eye or a right eye of the subject;
wherein the correction region has a two-dimensional double arc shape including a proximal arc and a distal arc, each of said proximal arc and distal arc face said lens center,
wherein the proximal arc extends between a first proximal end and a second proximal end, and said distal arc extends between a first distal end and a second distal end,
wherein the first proximal end and said first distal are connected by a first transverse line, and said second proximal end and second distal end is connected by a second transverse line,
wherein at least one point on said proximal arc has a proximal distance from said lens center that is between 4 mm and about 6 mm and at least one point on said distal arc has a distal distance from said lens center that is between about 13 mm and 14 mm; and
wherein a radial line connecting said first distal end to said lens center defines a first angle that is between 25° and 55° and a radial line connecting said second distal end to said lens center defines a second angle that is between 125° and 155°.
   2. The method of clause 1, wherein said proximal arc and said distal arc are parallel arcs.
   3. The method of clause 1 or 2, wherein at least one point on said proximal arc has a proximal distance of 4 mm.
   4. The method of any one of the preceding clauses, wherein at least one point on said distal arc has a distal distance of 14 mm.
   5. The method of any one of the preceding clauses, wherein said proximal arc has a fixed proximal distance along an entire length of said proximal arc.
   6. The method of any one of the preceding clauses, wherein said distal arc has a fixed distal distance along an entire length of said distal arc.
   7. The method of any one of the preceding clauses, wherein said first angle is equal or greater than 25°.
   8. The method of any one of the preceding clauses, wherein said second angle is equal or less than 155°.
   9. The method of any one of the preceding clauses, wherein said proximal arc has a fixed proximal distance of 4 mm along an entire length of said proximal arc and said distal arc has a fixed distal distance of 14 mm along an entire length of said distal arc.
   10. The method of any one of the preceding clauses, wherein said first angle is 25° and said second angle is 155°, such as illustrated in FIG. 2A.
   11. The method of any one of clauses 1 to 8, wherein said proximal arc has a fixed proximal distance of 4 mm along an entire length of said proximal arc and a first angle of 25° and said distal arc has a fixed distal distance of 14 mm along an entire length of said distal arc and a second angle of 155°, such as illustrated in FIG. 2A.
   12. The method of any one of clauses 1 to 8, wherein said proximal arc has a fixed proximal distance of 4 mm along an entire length of said proximal arc and a first angle greater than 25° and said distal arc has a fixed distal distance of 14 mm along an entire length of said distal arc and a second angle that is 155°, such as illustrated in FIG. 2C.
   13. The method of any one of clauses 1 to 8, wherein said proximal arc has a fixed proximal distance of 4 mm along an entire length of said proximal arc and a first angle that is 25° and said distal arc has a fixed distal distance of 14 mm along an entire length of said distal arc and a second angle that is smaller than 155°, such as illustrated in FIG. 2B.
   14. The method of any one of clauses 1 to 8, wherein said proximal arc has a fixed proximal distance of 4 mm along an entire length of said proximal arc and a first angle greater than 25° and said distal arc has a fixed distal distance of 14 mm along an entire length of said distal arc and a second angle that is smaller than 155°, such as illustrated in FIG. 2D.
   15. The method of any one of clauses 1 to 4, wherein at least one of said proximal arc and said distal arc has a variable distance from said lens center, such as illustrated in FIG. 2E.
   16. The method of any one of clauses 1 to 6, wherein said radial line connecting any one of said first distal end and second distal end is at an angle that is different from an angle defined by a proximal radial line connecting a respective first proximal end or second proximal end, such as illustrated in FIG. 2F.
   17. The method of any one of clause 1 to 14, wherein said radial line connecting said first distal end with said lens center converges with said first transverse line.
   18. The method of any one of the preceding clauses, wherein said device comprises a left lens and a right lens.
   19. The method of clause 18, wherein at least one first correcting element is placed at a left correction coordinate within a left correction region of said left lens, and at least one second correcting element is placed at a right correction coordinate within a right correction region of said right lens.
   20. The method of any one of the preceding clauses, wherein said device comprises two or more correcting elements in the correction region of said at least one lens.
   21. The method of any one of clauses 1 to 20, wherein said device comprises at least one correcting element placed at Ø=26°, R=4 on said left lens.
   22. The method of any one of clauses 1 to 20, wherein said device comprises at least one correcting element placed at Ø=75°, R=4 on said right lens.
   23. The method of any one of clauses 1 to 20, wherein said device comprises at least one correcting element placed at Ø=95°, R=13 on said left lens.
   24. The method of any one of clauses 1 to 20, wherein said device comprises at least one correcting element placed at Ø=61°, R=5 on the right lens.
   25. The method of any one of clauses 1 to 20, wherein said device comprises at least one correcting element placed at Ø=141°, R=5 on said right lens and at least one correcting element placed at Ø=26°, R=4 on said left lens.
   26. The method of any one of clauses 1 to 20, wherein said device comprises at least one correcting element placed at Ø=79°, R=13 on said left lens.
   27. The method of any one of clauses 1 to 20, wherein said device comprises at least one correcting element placed at Ø=102°, R=14 on said left lens.
   28. The method of any one of clauses 1 to 20, wherein said device comprises at least one correcting element placed Ø=111°, R=12 on said left lens.
   29. The method of any one of clauses 1 to 20, wherein said device comprises at least one correcting element placed at Ø=88°, R=13 on said left lens.
   30. The method of any one of clauses 1 to 20, wherein said device comprises at least one correcting element placed at Ø=65°, R=13 on said left lens.
   31. The method of any one of clauses 1 to 20, wherein said device comprises at least one correcting element placed at Ø=81°, R=14 on said right lens.
   32. The method of any one of clauses 1 to 20, wherein said device comprises at least one correcting element placed at Ø=76°, R=14 on said left lens.
   33. The method of any one of the preceding clauses, wherein said at least one correcting element is selected from the group consisting of a sticker, etch, color, engravement, digital mark, electronic mark, opaque mark, projection or the like placed or displayed in said correction region.
   34. The method of any one of the preceding clauses, wherein said device comprises two or more correcting elements that at least partially overlap.
   35. The method of any one of the preceding clauses, wherein said attention disorder is attention deficiency hyperactivity disorder (ADHD).
   36. A method for selecting a location of a correction coordinate in a device including at least one lens, the correction coordinate lies in the field of view of a subject, when the subject wears the device; the method comprising
identifying on said at least one lens a correction region,
   wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
   wherein said correction region has a two-dimensional double arc shape including a proximal arc and a distal arc, each of said proximal arc and distal arc face said lens center,
   wherein the proximal arc extends between a first proximal end and a second proximal end, and said distal arc extends between a first distal end and a second distal end,
   wherein the first proximal end and said first distal are connected by a first transverse line, and said second proximal end and second distal end is connected by a second transverse line,
   wherein said proximal arc has a proximal distance from said lens center that is between 4 mm and about 6 mm and said distal arc has a distal distance from said lens center that is between about 13 mm and 14 mm; and
   wherein a radial line connecting said first distal end to said lens center defines a first angle that is between 25° and 55° and a radial line connecting said second distal end to said lens center defines a second angle that is between 125° and 155°; and
selecting said location of said correction coordinate in said correction region for placing thereon a correcting element for improving attention performance in a subject having, a pre-existing, attention disorder.
   37. The method of clause 36, wherein said proximal arc and said distal arc are parallel arcs.
   38. The method of clause 36 or 37, wherein at least one point on said proximal arc has a proximal distance of 4 mm.
   39. The method of any one of clauses 36 to 38, wherein at least one point on said distal arc has a distal distance of 14 mm.
   40. The method of any one of clauses 36 to 39, wherein said proximal arc has a fixed proximal distance along an entire length of said proximal arc.
   41. The method of any one of clauses 36 to 40, wherein said distal arc has a fixed distal distance along an entire length of said distal arc.
   42. The method of any one of clauses 36 to 41, wherein said first angle is equal or greater than 25°.
   43. The method of any one of clauses 36 to 42, wherein said second angle is equal or less than 155°.
   44. The method of any one of clauses 36 to 43, wherein said proximal arc has a fixed proximal distance of 4 mm along an entire length of said proximal arc and said distal arc has a fixed distal distance of 14 mm along an entire length of said distal arc.
   45. The method of any one of clauses 36 to 44, wherein said first angle is 25° and said second angle is 155°, such as illustrated in FIG. 2A.
   46. The method of any one of clauses 36 to 45, wherein said proximal arc has a fixed proximal distance of 4 mm along an entire length of said proximal arc and a first angle of 25° and said distal arc has a fixed distal distance of 14 mm along an entire length of said distal arc and a second angle of 155°, such as illustrated in FIG. 2A.
   47. The method of any one of clauses 36 to 46, wherein said proximal arc has a fixed proximal distance of 4 mm along an entire length of said proximal arc and a first angle greater than 25° and said distal arc has a fixed distal distance of 14 mm along an entire length of said distal arc and a second angle that is 155°, such as illustrated in FIG. 2C.
   48. The method of any one of clauses 36 to 47, wherein said proximal arc has a fixed proximal distance of 4 mm along an entire length of said proximal arc and a first angle that is 25° and said distal arc has a fixed distal distance of 14 mm along an entire length of said distal arc and a second angle that is smaller than 155°, such as illustrated in FIG. 2B.
   49. The method of any one of clauses 36 to 48, wherein said proximal arc has a fixed proximal distance of 4 mm along an entire length of said proximal arc and a first angle greater than 25° and said distal arc has a fixed distal distance of 14 mm along an entire length of said distal arc and a second angle that is smaller than 155°, such as illustrated in FIG. 2D.
   50. The method of any one of clauses 36 to 49, wherein at least one of said proximal arc and said distal arc has a variable distance from said lens center, such as illustrated in FIG. 2E.
   51. The method of any one of clauses 36 to 41, wherein said radial line connecting any one of said first distal end and second distal end is at an angle that is different from an angle defined by a proximal radial line connecting a respective first proximal end or second proximal end, such as illustrated in FIG. 2F.
   52. The method of any one of clauses 36 to 51, wherein said radial line connecting said first distal end with said lens center converges with said first transverse line.
   53. The method of any one of clauses 36 to 52, wherein said device comprises a left lens and a right lens.
   54. The method of clause 53, wherein at least one first correcting element is placed at a left correction coordinate within a left correction region of said left lens, and at least one second correcting element is placed at a right correction coordinate within a right correction region of said right lens.
   55. The method of any one of clauses 36 to 54, wherein said device comprises two or more correcting elements in the correction region of said at least one lens.
   56. The method of any one of clauses 36 to 54, wherein said device comprises at least one correcting element placed at Ø=26°, R=4 on said left lens.
   57 The method of any one of clauses 36 to 54, wherein said device comprises at least one correcting element placed at Ø=75°, R=4 on said right lens.
   58. The method of any one of clauses 36 to 54, wherein said device comprises at least one correcting element placed at Ø=95°, R=13 on said left lens.
   59. The method of any one of clauses 36 to 54, wherein said device comprises at least one correcting element placed at Ø=61°, R=5 on the right lens.
   60. The method of any one of clauses 36 to 54, wherein said device comprises at least one correcting element placed at Ø=141°, R=5 on said right lens and at least one correcting element placed at Ø=26°, R=4 on said left lens.
   61. The method of any one of clauses 36 to 54, wherein said device comprises at least one correcting element placed at Ø=79°, R=13 on said left lens.
   62. The method of any one of clauses 36 to 54, wherein said device comprises at least one correcting element placed at Ø=102°, R=14 on said left lens.
   63. The method of any one of clauses 36 to 54, wherein said device comprises at least one correcting element placed Ø=111°, R=12 on said left lens.
   64. The method of any one of clauses 36 to 54, wherein said device comprises at least one correcting element placed at Ø=88°, R=13 on said left lens.
   65. The method of any one of clauses 36 to 54, wherein said device comprises at least one correcting element placed at Ø=65°, R=13 on said left lens.
   66. The method of any one of clauses 36 to 54, wherein said device comprises at least one correcting element placed at Ø=81°, R=14 on said right lens.
   67. The method of any one of clauses 36 to 54, wherein said device comprises at least one correcting element placed at Ø=76°, R=14 on said left lens.
   68. The method of any one of clauses 36 to 54, wherein said at least one correcting element is selected from the group consisting of a sticker, etch, color or opaque mark placed in said correction region.
   69. The method of any one of clauses 36 to 68, wherein said device comprises two or more correcting elements that at least partially overlap.
   70. The method of any one of clauses 36 to 68, wherein said attention disorder is attention deficiency hyperactivity disorder (ADHD).
   71. A method for converting an eyeglass into a device for improving performance of subject having a pre-existing, attention disorder, the method comprising:
selecting a location of at least one correction coordinate on at least one lens of said eyeglass, for placing thereon a correcting element, said selecting comprises at least the steps of
   identifying on said at least one lens a correction region,
      wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
      wherein said correction region has a two-dimensional double arc shape including a proximal arc and a distal arc, each of said proximal arc and distal arc face said lens center,
      wherein the proximal arc extends between a first proximal end and a second proximal end, and said distal arc extends between a first distal end and a second distal end;
      wherein the first proximal end and said first distal are connected by a first transverse line, and said second proximal end and second distal end is connected by a second transverse line;
      wherein said proximal arc has a proximal distance from said lens center that is between 4 mm and about 6 mm and said distal arc has a distal distance from said lens center that is between about 13 mm and 14 mm; and
      wherein a radial line connecting said first distal end to said lens center defines a first angle that is between 25° and 55° and a radial line connecting said second distal end to said lens center defines a second angle that is between 125° and 155°; and
   selecting said location of said correction coordinate in said correction region, the correction coordinate being within the subject's field of view when the eyeglass is worn by the subject; and
placing said at least one correcting element in the selected location in said correction coordinate.
   72. The method of clause 71, wherein said proximal arc and said distal arc are parallel arcs.
   73. The method of clause 71 or 72, wherein at least one point on said proximal arc has a proximal distance of 4 mm.
   74. The method of any one of clauses 71 to 73, wherein at least one point on said distal arc has a distal distance of 14 mm.
   75. The method of any one of clauses 71 to 74, wherein said proximal arc has a fixed proximal distance along an entire length of said proximal arc.
   76. The method of any one of clauses 71 to 75, wherein said distal arc has a fixed distal distance along an entire length of said distal arc.
   77. The method of any one of clauses 71 to 76, wherein said first angle is equal or greater than 25° 78. The method of any one of clauses 71 to 77, wherein said second angle is equal or less than 155°.
   79. The method of any one of clauses 71 to 78, wherein said proximal arc has a fixed proximal distance of 4 mm along an entire length of said proximal arc and said distal arc has a fixed distal distance of 14 mm along an entire length of said distal arc.
   80. The method of any one of clauses 71 to 79, wherein said first angle is 25° and said second angle is 155°, such as illustrated in FIG. 2A.
   81. The method of any one of clauses 71 to 79, wherein said proximal arc has a fixed proximal distance of 4 mm along an entire length of said proximal arc and a first angle of 25° and said distal arc has a fixed distal distance of 14 mm along an entire length of said distal arc and a second angle of 155°, such as illustrated in FIG. 2A.
   82. The method of any one of clauses 71 to 79, wherein said proximal arc has a fixed proximal distance of 4 mm along an entire length of said proximal arc and a first angle greater than 25° and said distal arc has a fixed distal distance of 14 mm along an entire length of said distal arc and a second angle that is 155°, such as illustrated in FIG. 2C.
   83. The method of any one of clauses 71 to 79, wherein said proximal arc has a fixed proximal distance of 4 mm along an entire length of said proximal arc and a first angle that is 25° and said distal arc has a fixed distal distance of 14 mm along an entire length of said distal arc and a second angle that is smaller than 155°, such as illustrated in FIG. 2B.
   84. The method of any one of clauses 71 to 79, wherein said proximal arc has a fixed proximal distance of 4 mm along an entire length of said proximal arc and a first angle greater than 25° and said distal arc has a fixed distal distance of 14 mm along an entire length of said distal arc and a second angle that is smaller than 155°, such as illustrated in FIG. 2D.
   85. The method of any one of clauses 71 to 76, wherein at least one of said proximal arc and said distal arc has a variable distance from said lens center, such as illustrated in FIG. 2E.
   86. The method of any one of clauses 71 to 85, wherein said radial line connecting any one of said first distal end and second distal end is at an angle that is different from an angle defined by a proximal radial line connecting a respective first proximal end or second proximal end, such as illustrated in FIG. 2E.
   87. The method of any one of clauses 71 to 84, wherein said radial line connecting said first distal end with said lens center converges with said first transverse line.
   88. The method of any one of clauses 71 to 84 or 87, wherein said radial line connecting said second distal end with said lens center converges with said second transverse line, such as illustrated in FIG. 2F.
   89. The method of any one of clauses 71 to 88, wherein said device comprises a left lens and a right lens.
   90. The method of clause 89, wherein at least one first correcting element is placed at a left correction coordinate within a left correction region of said left lens, and at least one second correcting element is placed at a right correction coordinate within a right correction region of said right lens.
   91. The method of any one of clauses 71 to 90, wherein said device comprises two or more correcting elements in the correction region of said at least one lens.
   92. The method of any one of clauses 71 to 91, wherein said device comprises at least one correcting element placed at Ø=26°, R=4 on said left lens.
   93. The method of any one of clauses 71 to 91, wherein said device comprises at least one correcting element placed at Ø=75°, R=4 on said right lens.
   94. The method of any one of clauses 71 to 91, wherein said device comprises at least one correcting element placed at Ø=95°, R=13 on said left lens.
   95. The method of any one of clauses 71 to 91, wherein said device comprises at least one correcting element placed at Ø=61°, R=5 on the right lens.
   96. The method of any one of clauses 71 to 91, wherein said device comprises at least one correcting element placed at Ø=141°, R=5 on said right lens and at least one correcting element placed at Ø=26°, R=4 on said left lens.
   97. The method of any one of clauses 71 to 91, wherein said device comprises at least one correcting element placed at Ø=79°, R=13 on said left lens.
   98. The method of any one of clauses 71 to 91, wherein said device comprises at least one correcting element placed at Ø=102°, R=14 on said left lens.
   99. The method of any one of clauses 71 to 91, wherein said device comprises at least one correcting element placed Ø=111°, R=12 on said left lens.
   100. The method of any one of clauses 71 to 91, wherein said device comprises at least one correcting element placed at Ø=88°, R=13 on said left lens.
   101. The method of any one of clauses 71 to 91, wherein said device comprises at least one correcting element placed at Ø=65°, R=13 on said left lens.
   102. The method of any one of clauses 71 to 91, wherein said device comprises at least one correcting element placed at Ø=81°, R=14 on said right lens.
   103. The method of any one of clauses 71 to 91, wherein said device comprises at least one correcting element placed at Ø=76°, R=14 on said left lens.
   104. The method of any one of clauses 71 to 91, wherein said at least one correcting element is selected from the group consisting of a sticker, etch, color, engravement, digital mark, electronic mark, opaque mark, projection or the like placed or displayed in said correction region.
   105. The method of any one of clauses 71 to 91, wherein said device comprises two or more correcting elements that at least partially overlap.
   106. The method of any one of clauses 71 to 105, wherein said attention disorder is attention deficiency hyperactivity disorder (ADHD).
   107. The method of any one of clauses 71 to 106, wherein said selecting of location of said correction coordinate comprises:
assessing attention performance of a subject;
determining boundaries of the correction region, and
selecting the correction coordinate along or within said boundaries, based on the subject's performance.
   108. The method of claim 107, wherein said assessing comprises placing a correcting element at said correction coordinate or simulating a correcting element.
   109. The method of claim 108, wherein said correcting element is a simulated correcting element.

It should be noted that the embodiments described herein are provided for illustrative purposes only and should not be construed as limiting the scope of the invention. Various modifications and variations can be made to the embodiments without departing from the spirit and scope of the invention. Therefore, any such variations, modifications, and equivalents that fall within the scope of the claims, as well as any improvements, refinements, or alternative embodiments that may be developed by those skilled in the art, should also be considered as being within the scope of the invention and are intended to be encompassed within this specification.

The following Examples are representative of techniques employed by the inventors in carrying out aspects of the presently disclosed subject matter. It should be appreciated that while these techniques are exemplary of some embodiments for the practice of the invention, those of skill in the art, in light of the present disclosure, will recognize that numerous modifications can be made without departing from the spirit and intended scope of the invention as defined in the specification as a whole.

### DESCRIPTION OF NON-LIMITING EXAMPLES

In each of the following non limiting Examples, each subject was administered with two eyeglasses, a first eyeglass (“Glasses #A”) with correcting elements within the correction region, and a second eyeglass (“Glasses #B) with correcting elements outside the defined correction region. The effect on the subject's attention performance was assessed using three acceptable tests as follows:

Conners' Continuous Performance Test-3 (CPT-3): The CPT-3 (Keith Conners, C., et al. (2018). Conners' Continuous Performance Test Third Edition. In: Kreutzer, J. S., DeLuca, J., Caplan, B. (eds) Encycloped1a of Clinical Neuropsychology. Springer, Cham.) is an objective test of attention and impulsivity that has been validated in individuals aged 8 years and above. The CPT-3 test is presented in a game-like format where 360 letters appear on the computer screen, one at a time, for approximately 250 ms. Respondents are required to press the space bar or click the mouse button when any letter except the letter “X” appears on the screen.

Trial Making Test (TMT)—The TMT test (Vakil, E., et al. (2009). Developmental changes in attention tests norms: Implications for the structure of attention. Child Neuropsychology, 15(1), 21-39) is a neuropsychological test of visual attention and task switching. It provides information about visual search speed, scanning, speed of processing, mental flexibility, as well as executive functioning. TMT test consists of two versions-version A requires the individual to draw a connecting line between consecutive circled digits, whereas, in version B, requires the individual to connect circles in numeric and alphabetic order, alternating between numbers and letters. The goal of the TMT test is to finish as quickly as possible, with the time taken to complete is being used as the primary performance measure.

Stroop test—The Stroop test (Stroop, J. R. (1935). Studies of interference in serial verbal reactions. Journal of Experimental Psychology, 18 (6), 643-662) is an attention test that measures selective attention, the ability to inhibit cognitive interference when two competing stimuli are presented simultaneously. In the current task version, the individual is asked to name the ink color of the colored box as fast and accurately as possible.

In the following non-limiting Examples, an improvement in attention performance was calculated in reference to the performance when wearing eyeglasses without any correcting element on the lenses. A difference of at least 2% in attention performance was considered an improvement.

### Example 1

A 13-year-old child, having a documented history of ADHD diagnosis by a certified clinician, was treated separately with two different glasses having correcting elements as follows.

Glasses 1A: having a correcting element at Ø=26°, R=4 on the left eye.

Glasses 1B: having a correcting element at Ø=163°, R=6 on the left eye.

Attention performance was assessed using TMT A Test. The TMT A test was performed before (wearing glasses without correcting elements) and after (wearing Glasses 1A or Glasses 1B). Table 1 provides the level of improvement (%).

TABLE 1
Improvement according to TMT A test

| Treatment | TMT A Improvement (%) |
|---|---|
| Glasses 1A: Ø = 26°, R = 4 on the left eye | 20% |
| Glasses 1B: Ø = 163º, R = 6 on the left eye | No improvement |

Table 1 shows that Glasses 1A provided a significant improvement in the subject's attention performance, while no improvement was concluded for Glasses 1B.

### Example 2

A 22.8-year-old female student, having a documented history of ADHD diagnosis by a certified clinician, was treated separately with two different glasses having correcting elements as follows.

Glasses 2A: having a correcting element at Ø=75°, R=4 on the right eye.

Glasses 2 having a correcting element at Ø=18°, R=7 on the left eye.

Attention performance was assessed using the three different tests: TMT A Test, CPT-3 test, and the Stroop test. The tests were performed before (wearing glasses without correcting elements) and after (wearing glasses with the correcting elements).

Table 2 provides the level of improvement (%) as determined by each of the tests.

TABLE 2
improvement by TMT A, CPT-3 and Stroop tests

| Treatment | TMT A Test | CPT-3 Test | Stroop Test |
|---|---|---|---|
| Glasses 2A | 33% | 9% | 13% |
| Glasses 2B | No improvement | No improvement | No improvement |

Table 2 shows that improvement was significant according to any one of the tests performed when the subject wore Glasses 2A, i.e. showing improvement in visual attention (TMT A), inattentiveness (CPT-3), and selective attention (Stroop) while no significant improvement was observed when the subject wore Glasses 2B.

### Example 3

A 21.5-year—old male, having a documented history of ADHD diagnosis by a certified clinician, was provided with two glasses having correcting elements.

Glasses 3A having a correcting element at Ø=95°, R=13 on the left eye

Glasses 3B having a correcting element at Ø=115°, R=15 on the left eye

Attention performance was assessed using TMT A Test and CPT-3 Test. The tests were performed before (wearing glasses without correcting elements) and after (wearing glasses with correcting elements).

Table 3 provides the level of improvement (%) based on the effect of each glass on attention performance.

TABLE 3
Improvement (%) according to TMT A Test and CPT-3 Test

| Treatment | TMT A | CPT-3 |
|---|---|---|
| Glasses 3A | 15% | 5% |
| Glasses 3B | No improvement | No improvement |

Table 3 shows that Glasses 3A provided improvement in both visual attention (TMT A test) and inattentiveness (CPT-3 test), while Glasses 3B showed no detectable improvement.

### Example 4

A 25.7-year-old woman, having a documented history of ADHD diagnosis by a certified clinician, was provided with two glasses having correcting elements.

Glasses 4A having a correcting element at Ø=61°, R=5 on the right eye

Glasses 4B having a correcting element at Ø=19°, R=7 on the left eye

Attention performance was assessed using TMT B Test and CPT-3 Test.

The tests were performed before (wearing glasses without correcting elements) and after (wearing glasses with correcting elements).

Table 4 provides improvement (%) according to each test.

TABLE 4
Improvement according to TMT B Test and CPT-3 Test

| Treatment | TMT B Improvement (%) | CPT-3 Improvement (%) |
|---|---|---|
| Glasses 4A | 21% | 7% |
| Glasses 4B | No improvement | No improvement |

Table 4 shows that Glasses 4A provide improvement in both visual attention (TMT B test) and inattentiveness (CPT-3 test), while Glasses 4B showed no detectable improvement.

### Example 5

A 14-year-old girl, having a documented history of ADHD diagnosis by a certified clinician, primarily inattention, was provided with two glasses having correcting elements.

Glasses 5A having a right correcting element at Ø=141°, R=5 on the right eye and a left correcting element at Ø=26°, R=4 on the left eye.

Glasses 5B having a right correcting element at Ø=73°, R=3 on the right eye and a left correcting element at Ø=120°, R=2 on the left eye.

Attention performance was assessed using CPT-3 Test. The CPT-3 test was performed before (wearing glasses without correcting elements) and after (wearing glasses with correcting elements).

Table 5 provides level of improvement (%) according to the CPT-3 test.

TABLE 5
Improvement (%) according to the CPT-3 test

| Treatment | CPT-3 Improvement (%) |
|---|---|
| Glasses 5A: | 2% |
| Ø = 141º, R = 5 on the right eye | |
| Ø = 26°, R = 4 on the left eye | |
| Glasses 5B: | No improvement |
| Ø = 73º, R = 3 on the right eye | |
| Ø = 120°, R = 2 on the left eye | |

Table 5 shows that Glasses 5A provide improvement in inattentiveness (CPT-3 test), while Glasses 5B showed no detectable improvement.

### Example 6

A 39.4-year-old female medical student, having a documented history of ADHD diagnosis by a certified clinician, was provided with two glasses having correcting elements.

Glasses 6A having a correcting element at Ø=79°, R=13 on the left eye

Glasses 6B having a correcting element at Ø=120°, R=15 on the left eye

Attention performance was assessed using the TMT A Test and the CPT-3 Test.

The tests were performed before (wearing glasses without correcting elements) and after (wearing glasses with correcting elements).

Table 6 provides level of improvement (%) according to the TMT A Test and the CPT-3 Test.

TABLE 6
Improvement according to CPT-3 Test and TMT APT-3 Test

| Treatment | CPT-3 Improvement (%) | TMT A Improvement (%) |
|---|---|---|
| Glasses 6A | 6% | 17% |
| Glasses 6B | No improvement | No improvement |

The improvement presented in Table 6 shows that Glasses 6A resulted in improving the subject's inattentiveness (CPT-3 test) and visual attention (TMT A test) while Glasses 6B showed no improvement.

### Example 7

A 26.6-year-old male, having a documented history of ADHD diagnosis by a certified clinician, was provided with two glasses having correcting elements.

Glasses 7A having a correcting element at Ø=102°, R=14 on the left eye

Glasses 7B having a correcting element at Ø=120°, R=16 on the left eye.

Attention performance was assessed using the TMT A Test and the CPT-3 Test. The tests were performed before (wearing glasses without correcting elements) and after (wearing glasses with correcting elements).

Table 7 provides level of improvement using each test

TABLE 7
Improvement according to TMT A Test and CPT-3 Test

| Treatment | CPT-3 Improvement (%) | TMT A Improvement (%) |
|---|---|---|
| Glasses 7A | 9% | 13% |
| Glasses 7B | No improvement | No improvement |

Table 7 shows significant improvement in the subject's inattentiveness (CPT-3 test) and visual attention (TMT A test) when the subject wore Glasses 7A, while no improvement was observed with Glasses 7B.

### Example 8

A 34.8-year-old male, having a documented history of ADHD diagnosis by a certified clinician, was provided with two glasses having correcting elements.

Glasses 8A having a correcting element at Ø=111°, R=12 on the left eye

Glasses 8B having a correcting element at Ø=129°, R=15 on the left eye

Attention performance was assessed using the Stroop Test and the CPT-3 Test.

The tests were performed before (wearing glasses without correcting elements) and after (wearing glasses with correcting elements).

Table 8 provides the level of improvement as determined by the two tests.

TABLE 8
Improvement according to CPT-3 Test and Stroop Test

| | | Stroop Test Improvement |
|---|---|---|
| Treatment | CPT-3 Improvement ( (%) | (%) |
| Glasses 8A | 13% | 23% |
| Glasses 8B | No improvement | No improvement |

Table 8 shows significant improvement in the subject's inattentiveness (CPT-3 test) and selective attention (Stroop test) when the subject wore Glasses 8A, while no improvement with Glasses 8B.

### Example 9

A 28.8-year-old female, having a documented history of ADHD diagnosis by a certified clinician, was treated separately with two different glasses having correcting elements as follows.

Glasses 9A: having a correcting element at Ø=88°, R=13 on the left eye.

Glasses 9 having a correcting element at Ø=65°, R=17 on the right eye.

Attention performance was assessed using the two different tests: TMT A Test, and the Stroop test. The tests were performed before (wearing glasses without correcting elements) and after (wearing glasses with the correcting elements).

Table 9 provides the level of improvement (%) as determined by each of the tests.

TABLE 9
improvement by TMT A, CPT-3 and Stroop tests

| Treatment | TMT A Test | Stroop Test |
|---|---|---|
| Glasses 9A | 19% | 3% |
| Glasses 9B | No improvement | No improvement |

Table 9 shows that improvement was observed according to both tests performed when the subject wore Glasses 9A, i.e. showing improvement in visual attention (TMT A), and selective attention (Stroop) while no significant improvement was observed when the subject wore Glasses 9B.

### Example 10

A 21.8-year-old female, having a documented history of ADHD diagnosis by a certified clinician, was treated separately with two different glasses having correcting elements as follows.

Glasses 10A: having a correcting element at Ø=65°, R=13 on the left eye.

Glasses 10B: having a correcting element at Ø=84°, R=15 on the right eye.

Attention performance was assessed using the Stroop test. The test was performed before (wearing glasses without correcting elements) and after (wearing glasses with the correcting elements).

Table 10 provides the level of improvement (%) as determined by the test.

TABLE 10
improvement by Stroop test

| Treatment | Stroop Test |
|---|---|
| Glasses 10A | 8% |
| Glasses 10B | No improvement |

Table 10 shows that Glasses 10A provided an improvement in the subject's attention performance, while no improvement was observed for Glasses 10B.

### Example 11

A 14-year-old male, having a documented history of ADHD diagnosis by a certified clinician, was provided with two glasses having correcting elements.

Glasses 11A having a correcting element at Ø=81°, R=14 on the right eye

Glasses 11B having a correcting element at Ø=62°, R=16 on the right eye

Attention performance was assessed using the TMT A Test.

The test was performed before (wearing glasses without correcting elements) and after (wearing glasses with correcting elements).

Table 11 provides the level of improvement (%) according to the TMT A Test.

TABLE 11
Improvement according to TMT A Test

| Treatment | TMT A Improvement (%) |
|---|---|
| Glasses 11A | 4% |
| Glasses 11B | No improvement |

The improvement presented in Table 11 shows that Glasses 11A resulted in improving the subject's visual attention (TMT A test) while Glasses 11B showed no improvement.

### Example 12

A 12-year-old female, having a documented history of ADHD diagnosis by a certified clinician, was provided with two glasses having correcting elements.

Glasses 12A having a correcting element at Ø=76°, R=14 on the left eye

Glasses 12B having a correcting element at Ø=58°, R=16 on the left eye

Attention performance was assessed using TMT B Test.

The test was performed before (wearing glasses without correcting elements) and after (wearing glasses with correcting elements).

Table 12 provides improvement (%) according to the TMT B test.

TABLE 12
Improvement according to TMT B Test

| Treatment | TMT B Improvement (%) |
|---|---|
| Glasses 12A | 25% |
| Glasses 12B | No improvement |

Table 12 shows that Glasses 12A provide improvement in visual attention (TMT B test), while Glasses 12B showed no detectable improvement.

### Example 13

A 16-year-old male, having a documented history of ADHD diagnosis by a certified clinician, was provided with two glasses having correcting elements.

Glasses 13A having a correcting element at Ø=139°, R=5 on the right eye

Glasses 13B having a correcting element at Ø=90°, R=3 on the right eye

Attention performance was assessed using TMT A and Stroop Tests.

The test were performed before (wearing glasses without correcting elements) and after (wearing glasses with correcting elements).

Table 13 provides improvement (%) according to the TMT A and Stroop tests.

TABLE 13
Improvement according to TMT A & Stroop Tests

| Treatment | TMT A Improvement (%) | Stroop Improvement (%) |
|---|---|---|
| Glasses 13A | 21% | 10% |
| Glasses 13B | No improvement | No improvement |

Table 13 shows that Glasses 13A provide improvement in attention performance (TMT A and Stroop tests), while Glasses 13B showed no detectable improvement.

### Example 14

A 47-year-old male, having a documented history of ADHD diagnosis by a certified clinician, was provided with two glasses having correcting elements.

Glasses 14A having a correcting element at Ø=97°, R=13 on the left eye

Glasses 14B having a correcting element at Ø=116°, R=15 on the left eye

Attention performance was assessed using TMT A and Stroop Tests.

The test were performed before (wearing glasses without correcting elements) and after (wearing glasses with correcting elements).

Table 14 provides improvement (%) according to the TMT A and Stroop tests.

TABLE 14
Improvement according to TMT A & Stroop Tests

| Treatment | TMT A Improvement (%) | Stroop Improvement (%) |
|---|---|---|
| Glasses 14A | 47% | 18% |
| Glasses 14B | No improvement | No improvement |

Table 14 shows that Glasses 14A provide improvement in attention performance (TMT A and Stroop tests), while Glasses 14B showed no detectable improvement.

### Example 15

A 19-year-old female, having a documented history of ADHD diagnosis by a certified clinician, was provided with two glasses having correcting elements.

Glasses 15A having a correcting element at Ø=148°, R=5 on the right eye

Glasses 15B having a correcting element at Ø=90°, R=3 on the right Figeye

Attention performance was assessed using TMT B Test.

The test was performed before (wearing glasses without correcting elements) and after (wearing glasses with correcting elements).

Table 15 provides improvement (%) according to the TMT B Test.

TABLE 15
Improvement according to TMT B Test

| Treatment | TMT B Improvement (%) |
|---|---|
| Glasses 15A | 40% |
| Glasses 15B | No improvement |

Table 15 shows that Glasses 15A provide improvement in attention performance (TMT B Test), while Glasses 15B showed no detectable improvement.

The non-limiting Examples provided above show that as long as the device had at least one correcting element within the boundaries of the double arc correction region there was an improvement in the subject's performance in at least one acceptable test, while no improvement was observed by the same test conditions when the subject wore a device with a correcting element outside the boundaries of the correction region.

1. A method for treating a subject having attention disorder, the method comprising:
administering, to the subject having the attention disorder, a device comprising at least one lens, and at least one correcting element disposed on the at least one lens,
wherein the at least one correcting element is disposed so as to be within the subject's field of view when the device is worn;
wherein the at least one correcting element is placed at a correction coordinate that is within a correction region;
wherein the correction region and the correction coordinate are each defined using a polar coordinate system measured from a lens center of a left eye or a right eye of the subject;
wherein the correction region has a two-dimensional double arc shape including a proximal arc and a distal arc, each of said proximal arc and distal arc face said lens center,
wherein the proximal arc extends between a first proximal end and a second proximal end, and said distal arc extends between a first distal end and a second distal end,
wherein the first proximal end and said first distal are connected by a first transverse line, and said second proximal end and second distal end is connected by a second transverse line,
wherein at least one point on said proximal arc has a proximal distance from said lens center that is between 4 mm and about 6 mm and at least one point on said distal arc has a distal distance from said lens center that is between about 13 mm and 14 mm; and
wherein a radial line connecting said first distal end to said lens center defines a first angle that is between 25° and 55° and a radial line connecting said second distal end to said lens center defines a second angle that is between 125° and 155°.

2. The method of claim 1, wherein said proximal arc and said distal arc are parallel arcs.

3. The method of claim 1, wherein at least one point on said proximal arc has a proximal distance of 4 mm and/or at least one point on said distal arc has a distal distance of 14 mm.

4. (canceled)

5. The method of claim 1, wherein said proximal arc has a fixed proximal distance along an entire length of said proximal arc and/or wherein said distal arc has a fixed distal distance along entire length of said distal arc.

6. (canceled)

7. The method of claim 1, wherein said first angle is equal or greater than 25° and/or wherein said second angle is equal or less than 155°.

8. (canceled)

9. The method of claim 1, wherein said proximal arc has a fixed proximal distance of 4 mm along an entire length of said proximal arc and said distal arc has a fixed distal distance of 14 mm along an entire length of said distal arc.

10. The method of claim 1, wherein said first angle is 25° and said second angle is 155°.

11. The method of claim 1, wherein said proximal arc has a fixed proximal distance of 4 mm along an entire length of said proximal arc and a first angle of 25° and said distal arc has a fixed distal distance of 14 mm along an entire length of said distal arc and a second angle of 155°.

12. The method of claim 1, wherein said proximal arc has a fixed proximal distance of 4 mm along an entire length of said proximal arc and a first angle greater than 25° and said distal arc has a fixed distal distance of 14 mm along an entire length of said distal arc and a second angle that is 155°.

13. The method of claim 1, wherein said proximal arc has a fixed proximal distance of 4 mm along an entire length of said proximal arc and a first angle that is 25° and said distal arc has a fixed distal distance of 14 mm along an entire length of said distal arc and a second angle that is smaller than 155°.

14. The method of claim 1, wherein said proximal arc has a fixed proximal distance of 4 mm along an entire length of said proximal arc and a first angle greater than 25° and said distal arc has a fixed distal distance of 14 mm along an entire length of said distal arc and a second angle that is smaller than 155°.

15. The method of claim 1, wherein at least one of said proximal arc and said distal arc has a variable distance from said lens center.

16. The method of claim 1, wherein said radial line connecting any one of said first distal end and second distal end is at an angle that is different from an angle defined by a proximal radial line connecting a respective first proximal end or second proximal end.

17. The method of claim 1, wherein said radial line connecting said first distal end with said lens center converges with said first transverse line and/or wherein said radial line connecting said second distal end with said lens center converges with said second transverse line.

18. (canceled)

19. The method of claim 1 to 18, wherein said device comprises a left lens and a right lens and at least one first correcting element is placed at a left correction coordinate within a left correction region of said left lens, and at least one second correcting element is placed at a right correction coordinate within a right correction region of said right lens.

20. (canceled)

21. (canceled)

22. The method of claim 1, wherein said device comprises at least one correcting element placed at the following correction coordinates:
at Ø=26°, R=4 on said left lens,
at Ø=75°, R=4 on said right lens;
at Ø=95°, R=13 on said left lens;
at Ø=61°, R=5 on said right lens;
at Ø=141°, R=5 on said right lens and at least one correcting element placed at Ø=26°, R=4 on said left lens;
at Ø=79°, R=13 on said left lens;
at Ø=102°, R=14 on said left lens;
at Ø=111°, R=12 on said left lens;
at Ø=88°, R=13 on said left lens;
at Ø=65°, R=13 on said left lens;
at Ø=81°, R=14 on said right lens;
at Ø=76°, R=14 on said left lens.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The method of claim 1, wherein said device comprises two or more correcting elements that at least partially overlap.

36. The method of claim 1, wherein said attention disorder is attention deficiency hyperactivity disorder (ADHD).

37. A method for selecting a location of a correction coordinate in a device including at least one lens, the correction coordinate lies in the field of view of a subject, when the subject wears the device; the method comprising
identifying on said at least one lens a correction region,
wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
wherein said correction region has a two-dimensional double arc shape including a proximal arc and a distal arc, each of said proximal arc and distal arc face said lens center,
wherein the proximal arc extends between a first proximal end and a second proximal end, and said distal arc extends between a first distal end and a second distal end,
wherein the first proximal end and said first distal are connected by a first transverse line, and said second proximal end and second distal end is connected by a second transverse line,
wherein said proximal arc has a proximal distance from said lens center that is between 4 mm and about 6 mm and said distal arc has a distal distance from said lens center that is between about 13 mm and 14 mm; and
wherein a radial line connecting said first distal end to said lens center defines a first angle that is between 25° and 55° and a radial line connecting said second distal end to said lens center defines a second angle that is between 125° and 155°; and
selecting said location of said correction coordinate in said correction region for placing thereon a correcting element for improving attention performance in a subject having attention disorder.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. A method for converting an eyeglass into a device for improving performance of subject having an attention disorder, the method comprising:
selecting a location of at least one correction coordinate on at least one lens of said eyeglass, for placing thereon a correcting element, said selecting comprises at least the steps of
identifying on said at least one lens a correction region,
wherein said correction region and said correction coordinate are each defined using a polar coordinate system measured from a lens center of the at least one lens, wherein the lens center is configured to be horizontally aligned with an optical center of a left eye or a right eye of the subject, when the device is worn by the subject;
wherein said correction region has a two-dimensional double arc shape including a proximal arc and a distal arc, each of said proximal arc and distal arc face said lens center,
wherein the proximal arc extends between a first proximal end and a second proximal end, and said distal arc extends between a first distal end and a second distal end;
wherein the first proximal end and said first distal are connected by a first transverse line, and said second proximal end and second distal end is connected by a second transverse line;
wherein said proximal arc has a proximal distance from said lens center that is between 4 mm and about 6 mm and said distal arc has a distal distance from said lens center that is between about 13 mm and 14 mm; and
wherein a radial line connecting said first distal end to said lens center defines a first angle that is between 25° and 55° and a radial line connecting said second distal end to said lens center defines a second angle that is between 125° and 155°; and
selecting said location of said correction coordinate in said correction region, the correction coordinate being within the subject's field of view when the eyeglass is worn by the subject; and
placing said at least one correcting element in the selected location in said correction coordinate.

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)
